(19) United States
(12) Patent Application Publication
(10) Pub. No.: US 2020/0348258 A1
(43) Pub. Date: Nov. 5, 2020

(54) SYSTEMS AND METHODS FOR FABRICATING AN INDIUM OXIDE FIELD-EFFECT TRANSISTOR

(71) Applicant: University of Southern California, Los Angeles, CA (US)

(72) Inventors: Chongwu Zhou, Los Angeles, CA (US); Xuan Cao, Los Angeles, CA (US); Yihang Liu, Los Angeles, CA (US); Qingzhou Liu, Los Angeles, CA (US); Fanqi Wu, Los Angeles, CA (US)

(21) Appl. No.: 16/933,765

(22) Filed: Jul. 20, 2020

Related U.S. Application Data
(62) Division of application No. 16/161,556, filed on Oct. 16, 2018.
(60) Provisional application No. 62/575,272, filed on Oct. 20, 2017.

(51) Int. Cl. G01N 27/414; H01L 29/66; H01L 29/24; G01N 33/543; H01L 21/02; H01L 21/443

(52) U.S. Cl. CPC G01N 27/4145; H01L 29/66969; H01L 29/24; G01N 33/5438; H01L 21/02565; H01L 21/443; G01N 27/4146; H01L 21/02603; H01L 21/02631

(57) ABSTRACT

Systems and methods for using an indium oxide field-effect transistor. A method includes applying phosphonic acid to a nanoribbon of the indium oxide field-effect transistor. The method also includes preparing the nanoribbon with capture antibodies corresponding to a biomarker. The method also includes applying a fluid sample containing at least one biomarker to the nanoribbon. The method also includes preparing the nanoribbon with secondary antibodies corresponding to the biomarker. The method also includes applying a protein solution to the nanoribbon. The method also includes detecting the presence of the at least one biomarker when a reactive solution is applied to the nanoribbon.

### CROSS-REFERENCE TO RELATED APPLICATIONS

This application is a divisional of U.S. patent application Ser. No. 16/161,556, titled “SYSTEMS AND METHODS FOR FABRICATING AN INDIUM OXIDE FIELD-EFFECT TRANSISTOR,” filed on Oct. 16, 2018, which claims priority to and the benefit of U.S. Provisional Application No. 62/575,272, titled “HIGHLY SENSITIVE AND QUICK DETECTION OF ACUTE MYOCARDIAL INFARCTION BIOMARKERS USING IN₂O₃ NANORIBBON BIOSENSORS FABRICATED USING SHADOW MASKS,” filed on Oct. 20, 2017, the entireties of each being hereby incorporated by reference herein.

### BACKGROUND

### 1. Field of the Invention

This specification relates to field-effect transistors.

### 2. Description of the Related Art

Indium oxide field-effect transistors have been shown to provide accurate results and quick turnaround times in detecting biomarkers within a patient's fluid sample. These properties make indium oxide field-effect transistors well suited for analyzing medical conditions that need urgent point-of-care (POC) medical attention, such as heart attacks. However, indium oxide field-effect transistors are generally made using a process that utilizes lithography. These processes inherently increase the cost and the fabrication time of indium oxide field-effect transistors which fiscally limits their widespread use as a diagnostic tool.

Accordingly, there is a need for a method of fabricating an indium oxide field-effect transistor without using lithography.

### SUMMARY OF THE INVENTION

In general, one aspect of the subject matter described in this specification is embodied in a method for fabricating indium oxide field-effect transistors. The method includes placing a first layer shadow mask onto a substrate, the first layer shadow mask having a first plurality of apertures. The method also includes depositing indium oxide through the first plurality of apertures and onto the substrate to form a plurality of indium oxide nanoribbons. The method also includes removing the first layer shadow mask. The method also includes placing a second layer shadow mask onto the substrate, the second layer shadow mask having a second plurality of apertures. The method also includes depositing a conductive material through the second plurality of apertures and onto the substrate to form a plurality of source and drain electrodes in electrical contact with the plurality of indium oxide nanoribbons. The method also includes removing the second layer shadow mask.

These and other embodiments may include one or more of the following features. The method may also include depositing an adhesion layer onto the substrate. The depositing of indium oxide may include using radio frequency sputtering. The depositing of the at least one conductive material may include using electron beam evaporation.

Each indium oxide nanoribbon within the plurality of indium oxide nanoribbons may be in electrical contact with only one source and drain electrode within the plurality of source and drain electrodes. Each source and drain electrode within the plurality of source and drain electrodes may be in electrical contact with only one indium oxide nanoribbon within the plurality of indium oxide nanoribbons. The deposition of indium oxide may be performed before the deposition of the conductive material. The deposition of indium oxide may be performed after the deposition of the conductive material.

At least a portion of each source and drain electrode within the plurality of source and drain electrodes may be deposited on top of at least a portion of each indium oxide nanoribbon within the plurality of indium oxide nanoribbons. Each source and drain electrode within the plurality of source and drain electrodes may adjoin a corresponding indium oxide nanoribbon within the plurality of indium oxide nanoribbons. At least a portion of each indium oxide nanoribbon within the plurality of indium oxide nanoribbons may be deposited on top of at least a portion of each source and drain electrode within the plurality of source and drain electrodes.

In another aspect, the subject matter is embodied in a method for using an indium oxide field-effect transistor. The method includes applying phosphonic acid to a nanoribbon of the indium oxide field-effect transistor. The method also includes preparing the nanoribbon with capture antibodies corresponding to a biomarker. The method also includes applying a fluid sample containing at least one biomarker to the nanoribbon. The method also includes preparing the nanoribbon with secondary antibodies corresponding to the biomarker. The method also includes applying a protein solution to the nanoribbon. The method also includes detecting the presence of the at least one biomarker when a reactive solution is applied to the nanoribbon.

### BRIEF DESCRIPTION OF THE DRAWINGS

The features and advantages of the embodiments of the present disclosure will become more apparent from the detailed description set forth below when taken in conjunction with the drawings. Naturally, the drawings and their associated descriptions illustrate example arrangements within the scope of the claims and do not limit the scope of the claims. Reference numbers are reused throughout the drawings to indicate correspondence between referenced elements.

FIG. 1A shows a perspective view of an array of indium oxide field-effect transistors according to an aspect of the invention.

FIG. 1B shows a close-up perspective view of an indium oxide field-effect transistor of FIG. 1A according to an aspect of the invention.

FIG. 1C shows a close-up side view of the indium oxide field-effect transistor of FIGS. 1A-1B according to an aspect of the invention.

FIGS. 2A-2I shows the fabrication of an array of indium oxide field-effect transistors according to an aspect of the invention.

FIGS. 3A-3I shows the fabrication of an array of indium oxide field-effect transistors according to an aspect of the invention.

FIG. 4A shows a close-up perspective view of an indium oxide field-effect transistor according to an aspect of the invention.

FIG. 4B shows a close-up side view of the indium oxide field-effect transistor of FIG. 4A according to an aspect of the invention.

FIG. 5A shows a close-up perspective view of an indium oxide field-effect transistor according to an aspect of the invention.

FIG. 5B shows a close-up side view of the indium oxide field-effect transistor of FIG. 5A according to an aspect of the invention.

FIG. 6 shows a flow diagram of an example fabrication process of an array of indium oxide field-effect transistors according to an aspect of the invention.

FIGS. 7A-7H shows the use of an indium oxide field-effect transistor according to an aspect of the invention.

FIGS. 8A-8F show electrical characterization of the indium oxide field-effect transistor according to an aspect of the invention.

FIGS. 9A-9F show experimental results using the indium oxide field-effect transistor according to an aspect of the invention.

FIGS. 10A-10C show experimental results using the indium oxide field-effect transistor and whole blood according to an aspect of the invention.

FIGS. 11A-11C demonstrate reuse of the indium oxide field-effect transistor according to an aspect of the invention.

### DETAILED DESCRIPTION

In the following detailed description, numerous specific details are set forth to provide an understanding of the present disclosure. It will be apparent, however, to one of ordinary skill in the art that elements of the present disclosure may be practiced without some of these specific details. In other instances, well-known structures and techniques have not been shown in detail to avoid unnecessarily obscuring the present disclosure.

Disclosed herein is a scalable and facile lithography-free method for fabricating highly uniform and sensitive In₂O₃ nanoribbon biosensor arrays. Fabrication with shadow masks as the patterning method instead of conventional lithography provides low-cost, time-efficient and high-throughput In₂O₃ nanoribbon biosensors without photoresist contamination. Combining with electronic enzyme-linked immunosorbent assay (ELISA) for signal amplification, the In₂O₃ nanoribbon biosensor arrays are optimized for early, quick and quantitative detection of cardiac biomarkers in diagnosis of acute myocardial infarction. Cardiac Troponin I (cTnI), Creatine kinase-MB (CK-MB) and B-type natriuretic peptide (BNP), which are commonly associated with heart attack and heart failure, are selected as the target biomarkers. The approach disclosed herein can detect label-free biomarkers for concentrations to a granularity of 1 pg/ml (cTnI), 0.1 ng/ml (CK-MB) and 10 pg/ml (BNP), which are all much lower than clinically relevant cut-off concentrations. In some embodiments, the sample-collection-to-result time is 45 minutes, and the reusability of the sensors has been demonstrated. With the demonstrated sensitivity, quick turnaround time, and reusability, the In₂O₃ nanoribbon biosensors disclosed herein allow early and quick diagnosis of acute myocardial infarction (AMI).

Every year approximately 5 million patients visit the emergency department because of chest pain symptoms, but only 10% of these patients experience acute myocardial infarction (AMI). If an initial electrocardiogram (ECG) assessment at the emergency department reveals a ST-segment elevation, the patient is placed at high risk for acute myocardial infarction (AMI), or heart attack, and the established medical procedures are administered to the patient. However, the ECG sensitivity may be as low as 50%, and patients who show no ST elevation can still be at high risk for unstable angina or non-ST segment elevation AMI. For this reason, cardiac biomarkers have become increasingly important for swift risk stratifying and diagnosing patients who may still need immediate treatment.

The effectiveness of the biomarkers to properly diagnose and triage chest pain patients is based on several factors. First, the test turnaround time should be short because early treatment of myocardial infarction is crucial to recovery. The American Heart Association has stated a recommended turn-around time of 60 minutes and a preferred turnaround time of 30 minutes from sample collection to result reporting. Second, obtaining the trend in the cardiac biomarker concentration in the hours after a patient's arrival is a crucial addition to the initial cardiac biomarker reading for accurate diagnosis. Current biomarker trends are collected through serial biomarker readings, such as testing at 0, 30, 60, and 90 minutes after patient arrival at the emergency department. Such rapid turnaround times are difficult to achieve in a central laboratory setting and is often aided by a point-of-care (POC) device. Additionally, multiple cardiac biomarkers testing may improve the diagnosis process of heart attack over single biomarker testing. The National Academy of Clinical Biochemistry has recommended testing for an early biomarker that elevates within the first 6 hours of chest pain in conjuncture with an AMI-specific biomarker that is increased in the blood even after 6 to 9 hours. Point-of-care platforms are ideal for multiple cardiac biomarker testing with rapid turnaround times, but current POC devices lack the sensitivity and high specificity of central laboratory biomarker testing. For POC devices to more effectively aid rapid decision making in both the emergency department and on the field, there is a need for further investigation of emerging sensor technology in order to bridge the performance gap between POC device and central laboratory testing for cardiac biomarkers.

Indium oxide (In₂O₃) field-effect transistors (FETs) have been shown to be real-time and label-free detectors with superb signal-to-noise ratio and the potential for integrated multiplexing. The rapid response time makes the In₂O₃ nanoribbon sensors especially advantageous for analyzing the first blood-drawn sample, from which rapid decisions can be made for the patients' treatment. The small device-to-device variation demonstrated previously can provide sufficient statistical confidence for calibrating cardiac biomarker concentrations. Furthermore, In₂O₃ nanoribbon sensors can provide quantitative analysis for a large detectable concentration range spanning at least 4 orders of magnitude and a detection limit in the picogram per milliliter range. This sensitivity can help to differentiate biomarker changes at each serial reading. Due to the electronic sensing, the final product enjoys facile interface and compactness while having the capability to integrate with other microfluidic and electronic functional groups, such as wireless data output. These unique properties make In₂O₃ nanoribbon sensors well suited for analyzing medical conditions such as heart attack which need urgent, point-of-care (POC) medical attention. It is highly important to develop a low-cost, time-efficient and scalable lithography-free process to produce In₂O₃ nanoribbon field effect transistors, which may generate broad impact to applications such as chemical sensing, protein detection, cancer diagnosis and prognosis, infectious disease diagnosis, biomedical research, and even thin film transistors for displays and macro electronics.

Disclosed herein is a lithography-free process for the fabrication of highly sensitive and scalable FET-based In₂O₃ nanoribbon biosensors. The nanoribbons are prepared by sputter-coating In₂O₃ through a shadow mask onto a substrate and have ribbon-like cross-section of approximately 16 nm in thickness and 25 μm in width and 500 μm in length, followed by metal electrode deposition through another shadow mask. The devices fabricated by shadow masks show good electrical performance in both ambient and aqueous environment, with the surfaces never exposed to undesirable chemicals like photoresist or e-beam resist. In addition, In₂O₃ nanoribbon devices also show good performance in pH sensing experiments. Through all the sensing experiments, it has been demonstrated that In₂O₃ nanoribbon biosensors fabricated using shadow masks can be used to quantitatively detect 3 cardiac biomarkers within the concentrations relevant to clinical diagnosis with the turnaround time being approximately 45 minutes. Tests using spiked cardiac biomarkers in diluted human blood were further demonstrated. Lastly, by first applying regeneration buffer to the used sensor surface to anti-bond the antigen-antibody conjugation and then repeating the sensing experiments, the reusability of the In₂O₃ nanoribbon biosensors with very small variation of each sensing results was demonstrated.

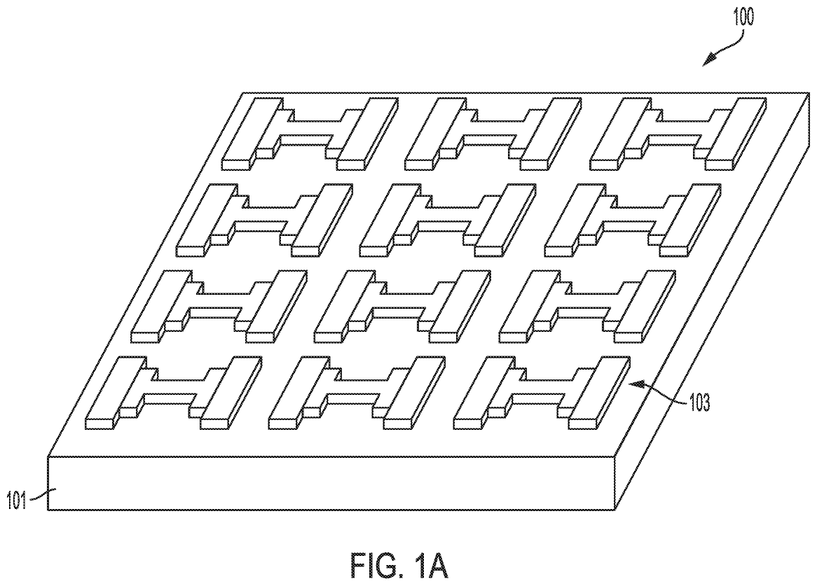

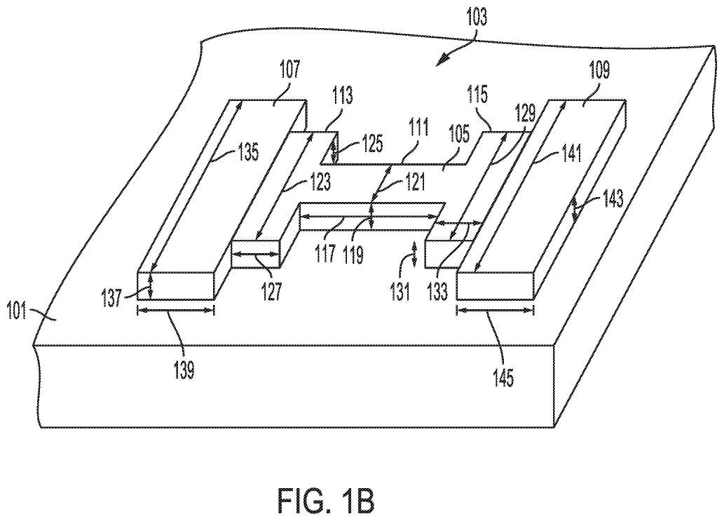

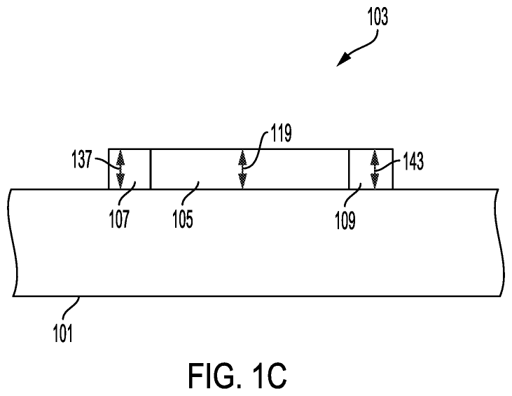

FIG. 1A shows a perspective view of an array of indium oxide field-effect transistors 100. The array of indium oxide field-effect transistors 100 includes indium oxide field-effect transistors 103 arranged in a matrix. The matrix is depicted a four by three matrix, but any number of rows and columns may form the matrix. The array of indium oxide field-effect transistors 100 may be arranged in various other formations according to various embodiments.

Each indium oxide field-effect transistor 103 is formed on a substrate 101. The substrate 101 may be formed from various materials including silicon, polyethylene terephthalate (PET), and glass. The substrate 101 may further include a base and an additional coating. For example, the substrate may be formed from a base of silicon with a coating of silicon oxide.

In some embodiments, there may be 28 groups of In₂O₃ nanoribbon FETs patterned over a 3-inch wafer using shadow masks, with each group containing five FET devices. The nanoribbons may be identical with very clear edges. In these embodiments, the channel width and length are 25 μm and 500 μm, respectively. Furthermore, In₂O₃ nanoribbons are smooth with 16 nm thickness.

FIG. 1B shows an indium oxide field-effect transistor 103 from FIG. 1A. The indium oxide field-effect transistor 103 includes a nanoribbon 105, a source electrode 107, and a drain electrode 109.

The nanoribbon 105 includes a central section 111, a first end section 113, and a second end section 115. The nanoribbon is composed of indium oxide. The central section 111 has a length 117, a thickness 119, and a width 121. In some embodiments, the width 121 may be 25 micrometers. The first end section 113 has a length 123, a thickness 125, and a width 121. The second end section 115 similarly has a length 129, a thickness 131, and a length 133. In some embodiments, the length 117, length 127, and length 133 may add up to a total length of around 500 micrometers.

The thickness 119 of the central section 111, the thickness 125 of the first end section 113, and the thickness 131 of the second end section 115 may all be the same (shown in FIG. 1C). In some embodiments, the thickness 119, thickness 125, and thickness 131 may be around 16 nanometers. However, other configurations may be used interchangeably according to various embodiments. The central section 111, the first end section 113, and the second end section 115 are depicted as having a rectangular cross section. However, other cross sectional geometries may be used, for example, a truncated cylinder, a rounded square, or a rounded rectangle.

The length 123 of the first end section 113 and the length 129 of the second end section 115 are depicted as being longer than the width 121 of the central section 111. It may be desirable for the lengths 123 and 129 to be longer than the width 121 in order to ensure a solid and/or reliable electrical connection with the source electrode 107 and the drain electrode 109. In other embodiments, the lengths 123 and 129 may be the same as the width 121 for simplicity of manufacture and/or to fit more indium oxide field-effect transistors on a given substrate 101.

The source electrode 107 has a length 135, a thickness 137, and a width 139. The drain electrode 109 has a length 141, a thickness 143, and a width 145. The thicknesses 137, 125, 131, and 143 may all be the same (shown in FIG. 1C). In some embodiments, the thicknesses 137, 125, 131, and 143 may be around 16 nanometers. However, other configurations may be used interchangeably according to various embodiments. The source electrode 107 and the drain electrode 109 are depicted as having a rectangular cross section. However, other cross sectional geometries may be used, for example, a truncated cylinder, a rounded square, or a rounded rectangle.

The source electrode 107 and the drain electrode 109 are composed of a conductive material. The conductive material may be any element or composition that is capable of electrical conduction. For example, the source electrode 107 and the drain electrode 109 may be composed of gold or a gold alloy.

The length 135 of the source electrode 107 and the length 141 of the drain electrode 109 are depicted as being longer than the lengths 123 and 129 of the first end section 113 and the second end section 115 respectively. It may be desirable for the lengths 135 and 141 to be longer in order to ensure a solid and/or reliable electrical connection with the first end section 113 and the second end section 115. In other embodiments, the lengths 135 and 141 may be the same as the lengths 123 and 133 for simplicity of manufacture and/or to fit more indium oxide field-effect transistors on a given substrate 101.

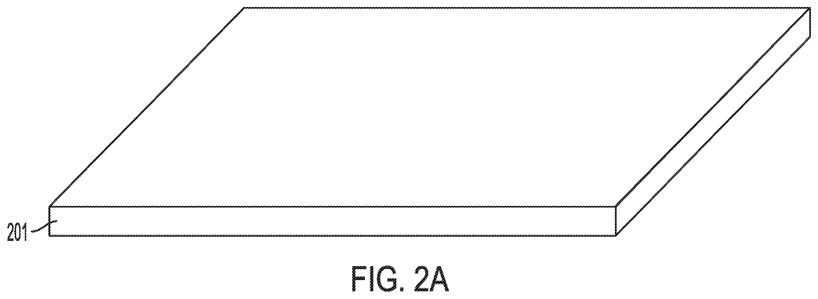

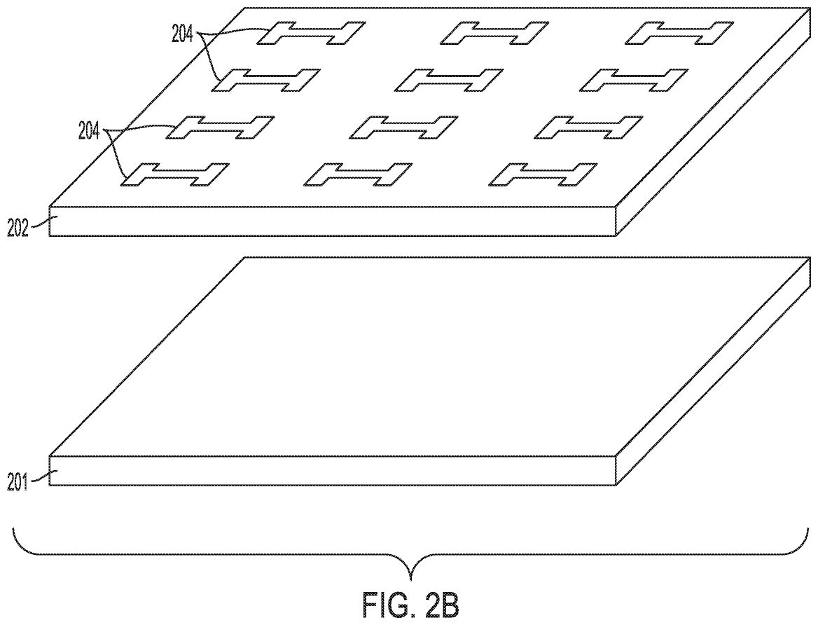

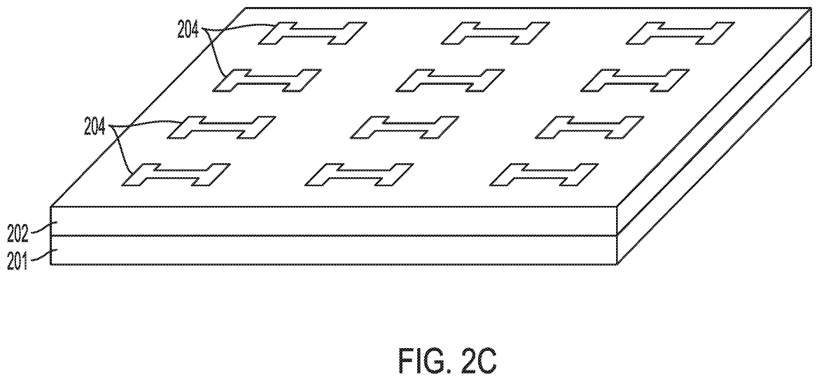

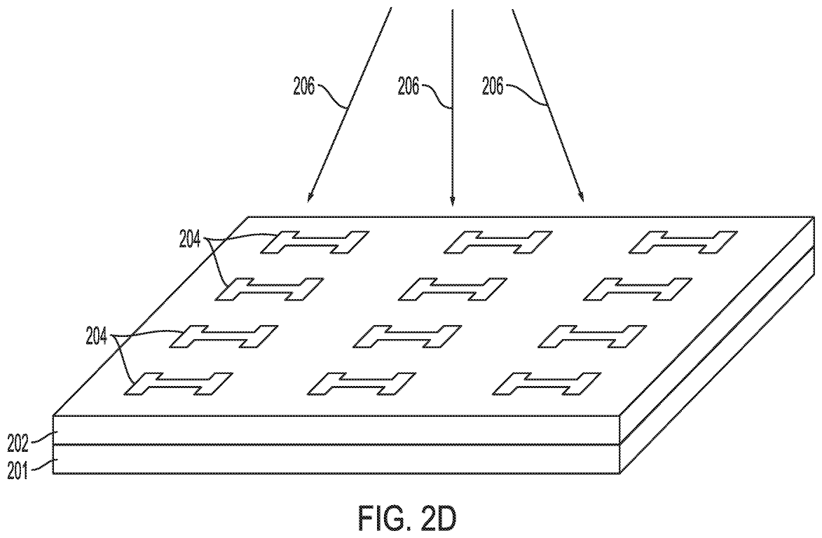

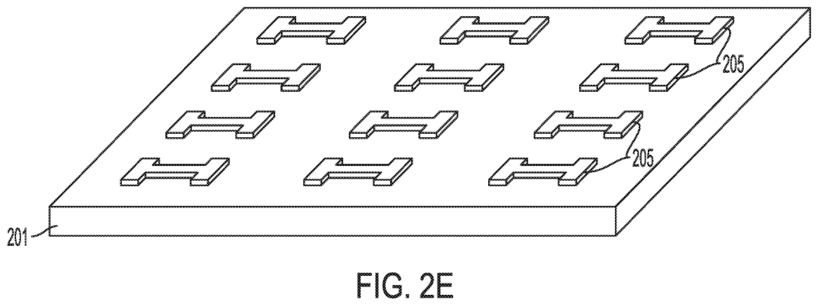

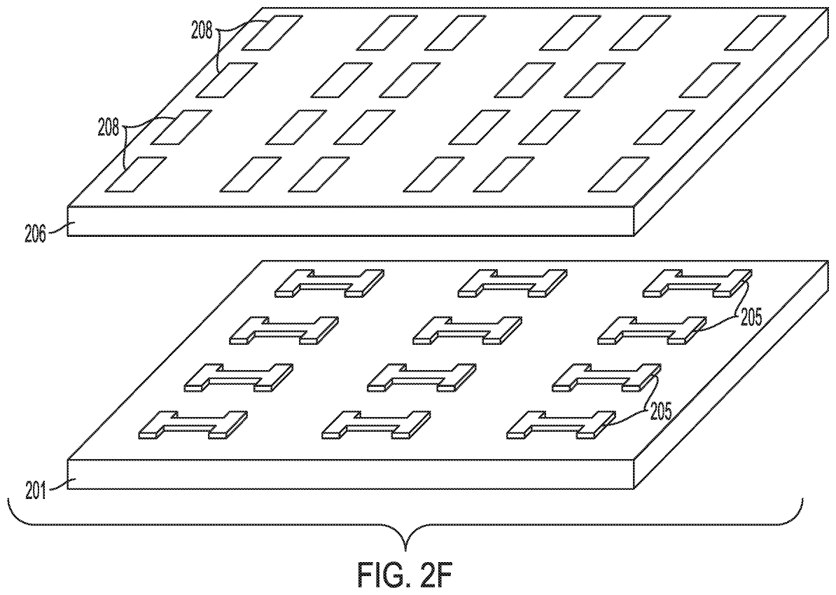

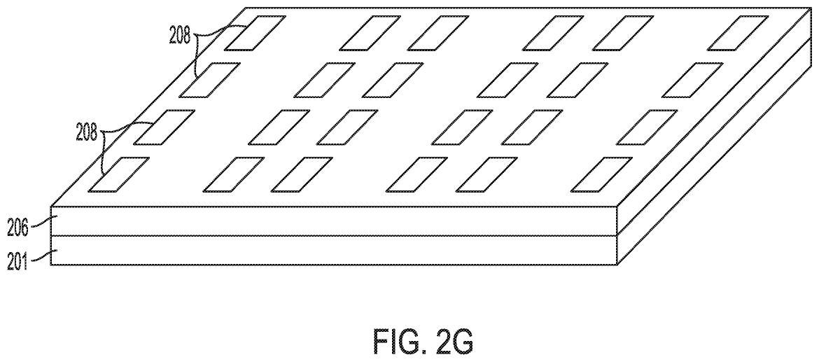

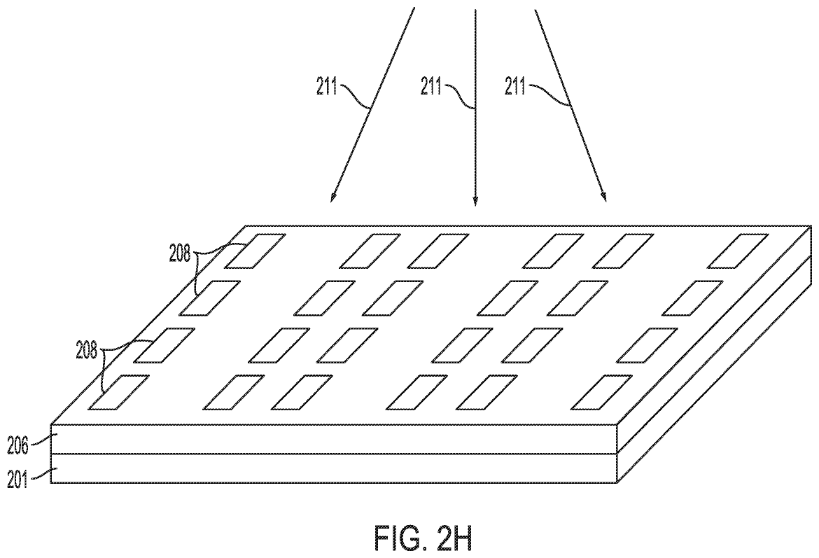

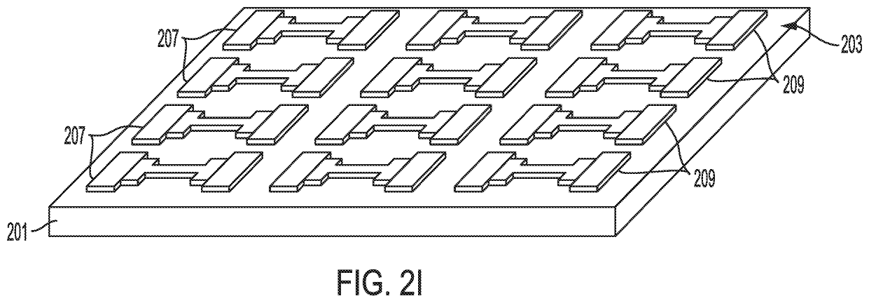

FIGS. 2A-2I shows the fabrication of an array of indium oxide field-effect transistors 203. The indium oxide field-effect transistors 203 are similar to the indium oxide field-effect transistors 103, and like parts are numbers similarly.

FIG. 2A shows a substrate 201 before any coatings or depositions have been performed on its surface. The substrate 201 may be composed of silicon, PET, or glass. In some embodiments, the substrate may be composed of silicon with a layer of silicon oxide on its surface.

FIG. 2B shows a first layer shadow mask 202 being positioned over the substrate 201. The first layer shadow mask 202 includes a plurality of apertures 204. The plurality of apertures are in the shape and/or form of the nanoribbons 205 (shown in FIG. 2E). The number of apertures within the plurality of apertures 204 may correspond directly with the number of nanoribbons to be deposited on the substrate 201. For example, if 12 nanoribbons are to be deposited there may be 12 apertures within the plurality of apertures. In some embodiments, the number of apertures within the plurality of apertures 204 may be a multiple of the number of nanoribbons to be deposited on the substrate 201. For example, if 12 nanoribbons are to be deposited there may be 4 apertures within thin the plurality of apertures. The first layer shadow mask 202 would be used 3 times to form 12 total nanoribbons on the substrate 201.

FIG. 2C shows the first layer shadow mask 202 being placed on top of the substrate 201. The first layer shadow mask 202 may be in direct contact with the substrate 201 when it is placed on top. In other embodiments, the first layer shadow mask 202 may be separated from the substrate by a distance when the first layer shadow mask 202 is placed on top of the substrate 201.

FIG. 2D shows indium oxide 211 being deposited over the first layer shadow mask 202 through the first plurality of apertures 204 and onto the substrate 201 to form the plurality of nanoribbons 205 (shown in FIG. 2E). The deposition of the indium oxide 211 may be performed using radio frequency sputtering. However, other methods of coating may be used, for example, cathodic arc deposition, pulsed laser deposition, direct ion beam deposition, plasma-enhanced chemical vapor deposition, chemical vapor deposition, or ion beam sputtering. After the indium oxide 211 is deposited onto the substrate 201, the first layer shadow mask 202 is removed.

FIG. 2F shows a second layer shadow mask 206 being positioned over the substrate 201. The second layer shadow mask 206 includes a second plurality of apertures 208. The plurality of apertures are in the shape and/or form of the plurality of source electrodes 207 and drain electrodes 209 (shown in FIG. 2I). The number of apertures within the second plurality of apertures 208 may correspond directly with the number of source electrodes 207 and drain electrodes 209 to be deposited on the substrate 201. For example, if 12 sets of source electrodes 207 and drain electrodes 209 are to be deposited there may be 24 apertures within the second plurality of apertures 208. In some embodiments, the number of apertures within the second plurality of apertures 208 may be a multiple of the number of sets of source electrodes 207 and drain electrodes 209 to be deposited on the substrate 201. For example, if 12 sets of source electrodes 207 and drain electrodes 209 are to be deposited, there may be 8 apertures within the second plurality of apertures 208. The second layer shadow mask 206 would be used 3 times to form 12 sets of source electrodes 207 and drain electrodes 209 on the substrate 201.

FIG. 2G shows the second layer shadow mask 206 being placed on top of the substrate 201. The second layer shadow mask 206 may be in direct contact with the substrate 201 when it is placed on top. In other embodiments, the second layer shadow mask 206 may be separated from the substrate 201 by a distance when the second layer shadow mask 206 is placed on top of the substrate 201.

FIG. 2H shows a conductive material 210 being deposited over the second layer shadow mask 206 through the second plurality of apertures 208 and onto the substrate 201 to form the plurality of source electrodes 207 and drain electrodes 209 (shown in FIG. 2I). The deposition of the conductive material 210 may be performed using electron beam evaporation. However, other methods of coating may be used, for example, cathodic arc deposition, pulsed laser deposition, direct ion beam deposition, plasma-enhanced chemical vapor deposition, chemical vapor deposition, or ion beam sputtering.

In some embodiments, an adhesion layer may be deposited onto the substrate 201 before the conductive material 210 is deposited in order to enhance adhesion of the conductive material to the substrate 201. After the conductive material 210 is deposited onto the substrate 201, the second layer shadow mask 206 is removed. FIG. 2I shows the manufactured indium oxide field-effect transistors 203 on the substrate 201.

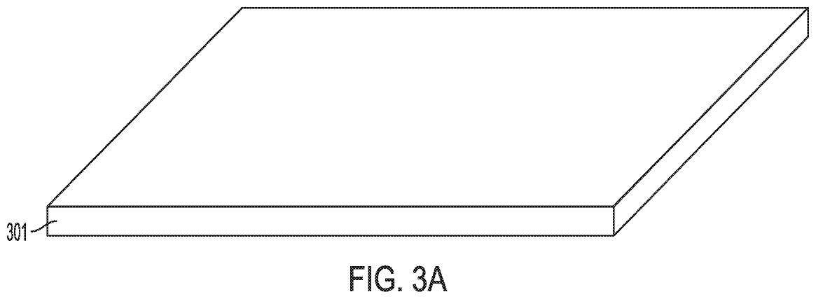

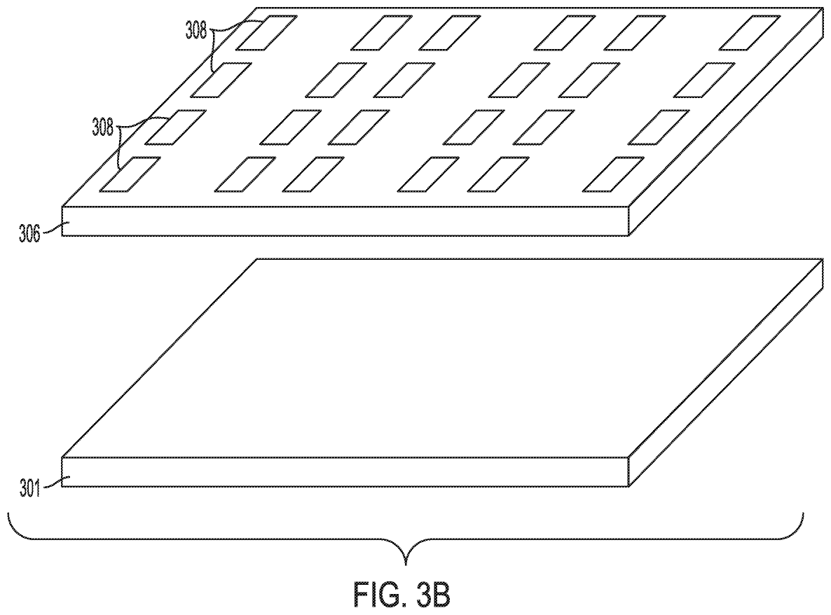

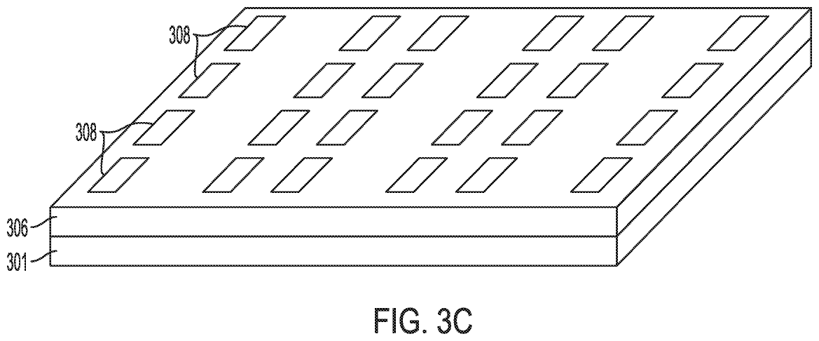

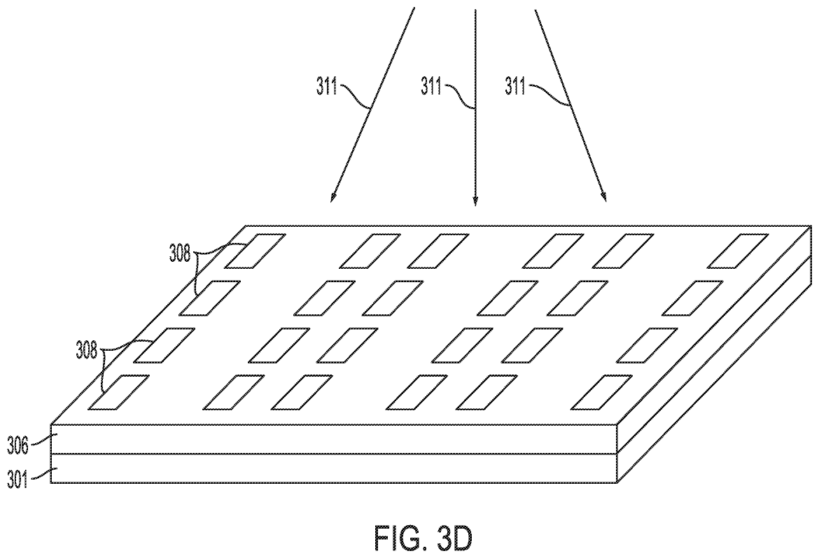

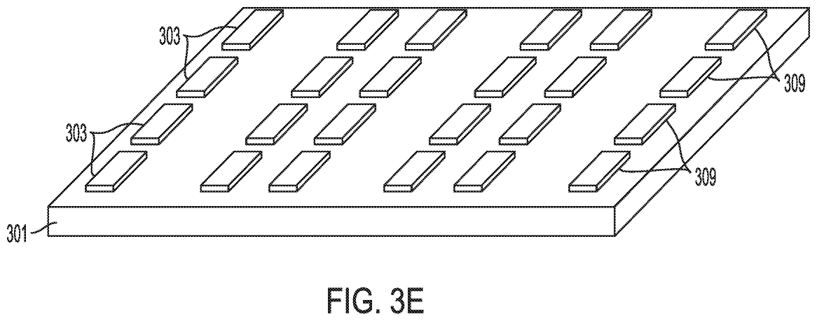

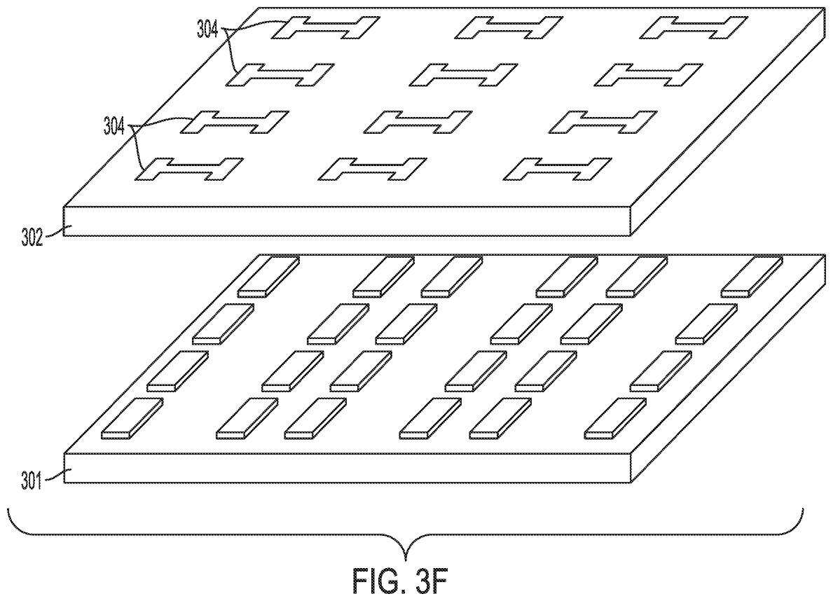

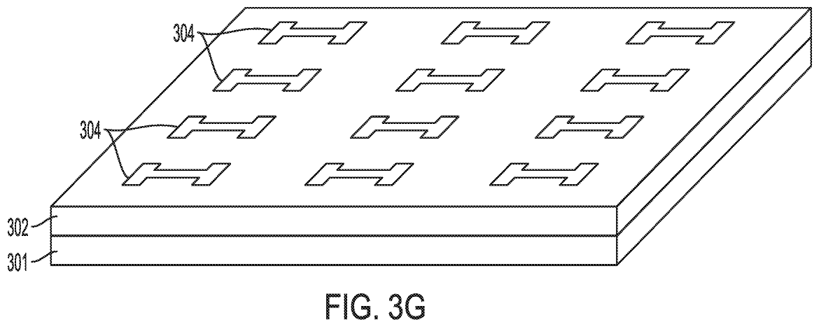

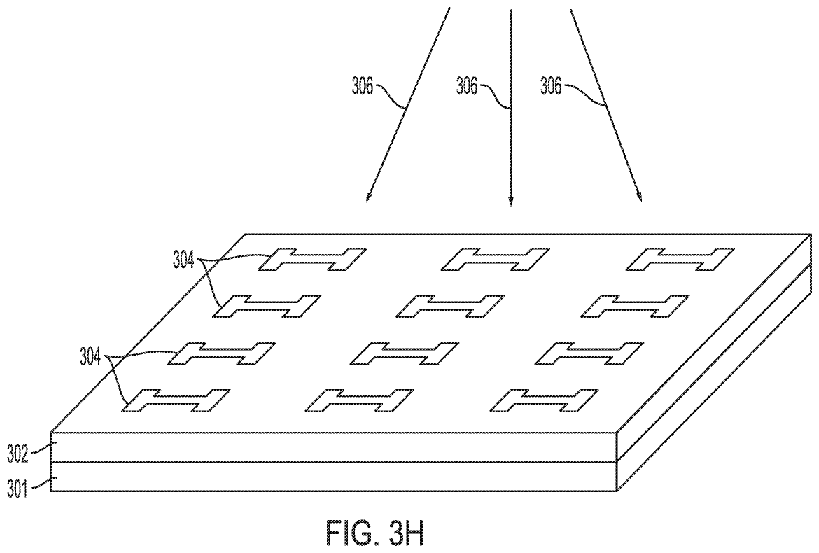

FIGS. 3A-3I show the fabrication of an array of indium oxide field-effect transistors 303. The indium oxide field-effect transistors 303 are similar to the indium oxide field-effect transistors 103 and 203, and like parts are numbers similarly.

FIG. 3A shows a substrate 301 before any coatings or depositions have been performed on its surface. The substrate 301 may be composed of silicon, PET, or glass. In some embodiments, the substrate may be composed of silicon with a layer of silicon oxide on its surface.

FIG. 3B shows a first layer shadow mask 306 being positioned over the substrate 301. The first layer shadow mask 306 includes a first plurality of apertures 308. The plurality of apertures are in the shape and/or form of source electrodes 307 and drain electrodes 309 (shown in FIG. 3I). The number of apertures within the first plurality of apertures 308 may correspond directly with the number of source electrodes 307 and drain electrodes 309 to be deposited on the substrate 301. For example, if 12 sets of source electrodes 307 and drain electrodes 309 are to be deposited, there may be 24 apertures within the first plurality of apertures 308. In some embodiments, the number of apertures within the first plurality of apertures 308 may be a multiple of the number of sets of source electrodes 307 and drain electrodes 309 to be deposited on the substrate 301. For example, if 12 sets of source electrodes 307 and drain electrodes 309 are to be deposited, there may be 8 apertures within the first plurality of apertures 308, and the first layer shadow mask 306 may be used 3 times to form 12 sets of source electrodes 307 and drain electrodes 309 on the substrate 301.

FIG. 3C shows the first layer shadow mask 306 being placed on top of the substrate 301. The first layer shadow mask 306 may be in direct contact with the substrate 301 when it is placed on top. In other embodiments, the first layer shadow mask 306 may be separated from the substrate 301 by a distance when the first layer shadow mask 306 is placed on top of the substrate 301.

FIG. 3D shows a conductive material 310 being deposited over the first layer shadow mask 306 through the first plurality of apertures 308 and onto the substrate 301 to form the plurality of source electrodes 307 and drain electrodes 309 (shown in FIG. 3I). The deposition of the conductive material 310 may be performed using electron beam evaporation. However, other methods of coating may be used, for example, cathodic arc deposition, pulsed laser deposition, direct ion beam deposition, plasma-enhanced chemical vapor deposition, chemical vapor deposition, or ion beam sputtering.

In some embodiments, an adhesion layer may be deposited onto the substrate 301 before the conductive material 310 is deposited in order to enhance adhesion of the conductive material to the substrate 301. After the conductive material 310 is deposited onto the substrate 301, the first layer shadow mask 306 is removed. FIG. 3E shows the source electrodes 207 and the drain electrodes 209 on the substrate 301.

FIG. 3F shows a second layer shadow mask 302 being positioned over the substrate 301. The second layer shadow mask 302 includes a plurality of apertures 304. The plurality of apertures are in the shape and/or form of the nanoribbons 305 (shown in FIG. 3I). The number of apertures within the plurality of apertures 304 may correspond directly with the number of nanoribbons to be deposited on the substrate 301. For example, if 12 nanoribbons are to be deposited there may be 12 apertures within the plurality of apertures. In some embodiments, the number of apertures within the plurality of apertures 304 may be a multiple of the number of nanoribbons to be deposited on the substrate 301. For example, if 12 nanoribbons are to be deposited there may be 4 apertures within thin the plurality of apertures. The second layer shadow mask 302 would be used 3 times to form 12 total nanoribbons on the substrate 301.

FIG. 3G shows the second layer shadow mask 302 being placed on top of the substrate 301. The second layer shadow mask 302 may be in direct contact with the substrate 301 when it is placed on top. In other embodiments, the second layer shadow mask 302 may be separated from the substrate by a distance when the second layer shadow mask 302 is placed on top of the substrate 301.

FIG. 3H shows indium oxide 311 being deposited over the second layer shadow mask 302 through the second plurality of apertures 304 and onto the substrate 301 to form the plurality of nanoribbons 305 (shown in FIG. 3I). The deposition of the indium oxide 311 may be performed using radio frequency sputtering. However, other methods of coating may be used, for example, cathodic arc deposition, pulsed laser deposition, direct ion beam deposition, plasma-enhanced chemical vapor deposition, chemical vapor deposition, or ion beam sputtering. After the indium oxide 311 is deposited onto the substrate 301, the second layer shadow mask 302 is removed.

FIG. 3I shows the manufactured indium oxide field-effect transistors 303 on the substrate 301.

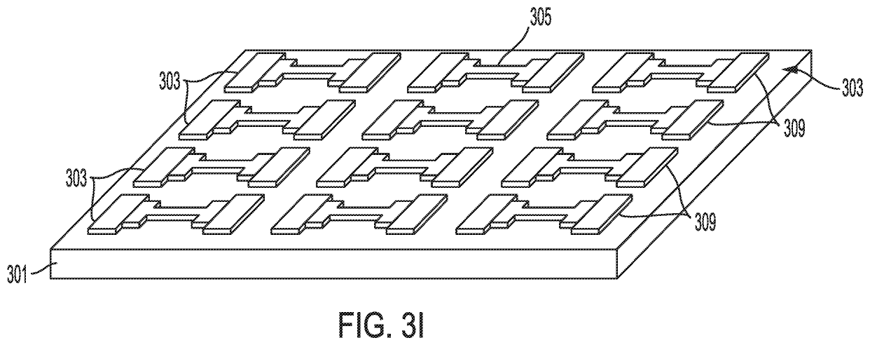

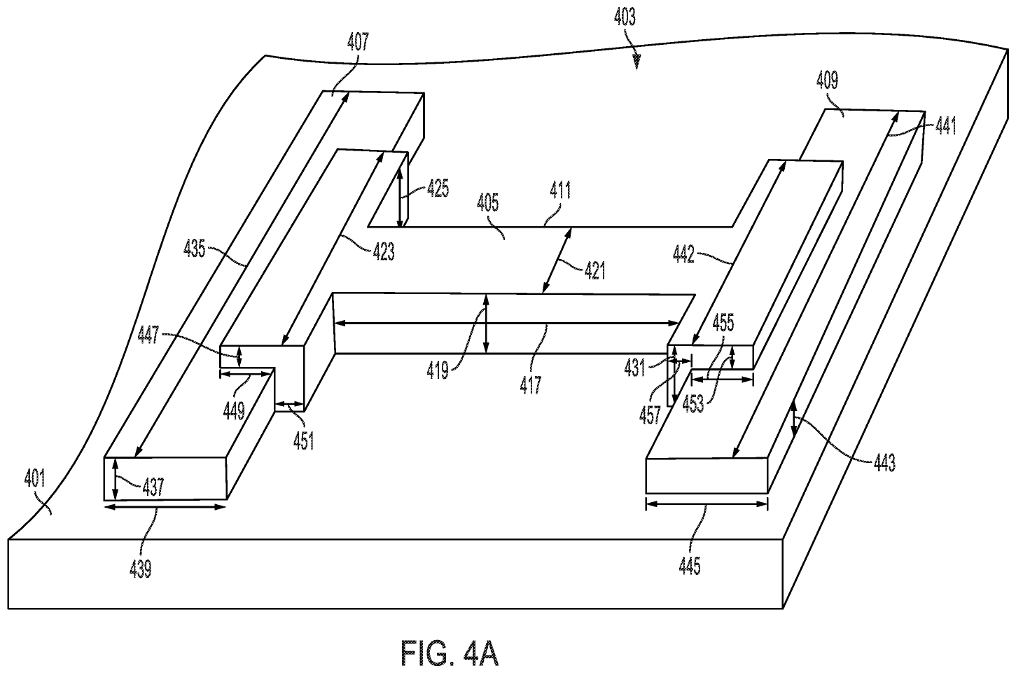

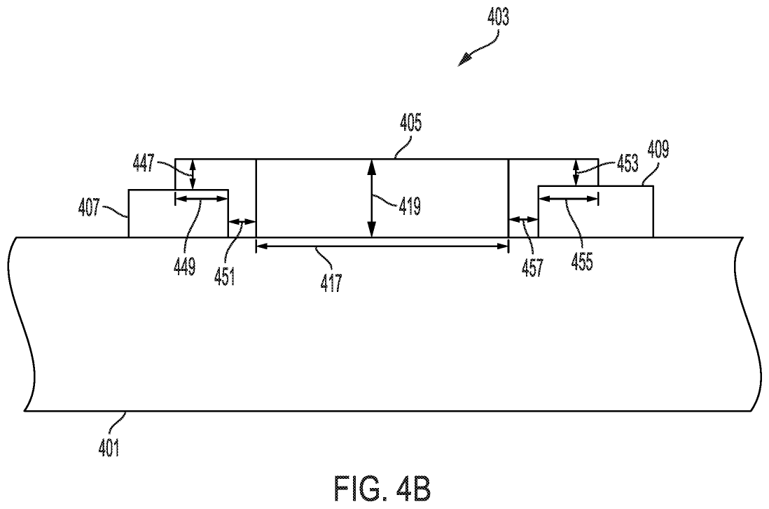

FIGS. 4A-4B show an alternate embodiment of an indium oxide field-effect transistor which may be used with the systems described herein. FIG. 4A shows a perspective view and FIG. 4B shows a side view. The indium oxide field-effect transistor 403 includes a nanoribbon 405, a source electrode 407, and a drain electrode 409.

The nanoribbon 405 includes a central section 411. The nanoribbon 405 is composed of indium oxide. The central section 411 has a length 417, a thickness 419, and a width 421. The two ends of the central section 411 may overlap with the source electrode 407 and the drain electrode 409. A first end of the central section which overlaps with the source electrode 407 has a length 423 and a thickness 425. The first end may have an overlapping width 449 and a non-overlapping width 451. The overlapping portion of the first end may have a height 447, which is the distance between the plane defined by the top surface of the central section 411 and the plane defined by the top surface of the source electrode 407. The second end of the central section which overlaps with the drain electrode 409 similarly has a length 442 and a thickness 431. The second end may have an overlapping width 455 and a non-overlapping width 457. The overlapping portion of the second end may have a height 453, which is the distance between the plane defined by the top surface of the central section 411 and the plane defined by the top surface of the drain electrode 409.

The thickness 419 of the central section 411, the thickness 425 of the first end, and the thickness 431 of the second end may all be the same.

The lengths 423 and 442 are depicted as being longer than the width 421 of the central section 411. It may be desirable for the lengths 423 and 442 to be longer than the width 421 in order to ensure a solid and/or reliable electrical connection with the source electrode 407 and the drain electrode 409. In other embodiments, the lengths 423 and 442 may be the same as the width 421 for simplicity of manufacture and/or to fit more indium oxide field-effect transistors on a given substrate 401.

The source electrode 407 has a length 435, a thickness 437, and a width 439. The drain electrode 409 has a length 441, a thickness 443, and a width 445. The thicknesses 437 and 443 may be the same. However, other configurations may be used interchangeably according to various embodiments. The source electrode 407 and the drain electrode 409 are depicted as having a rectangular cross-section. However, other cross-sectional geometries may be used, for example, a truncated cylinder, a rounded square, or a rounded rectangle.

The source electrode 407 and the drain electrode 409 are composed of a conductive material. The conductive material may be any element or composition that is capable of electrical conduction. For example, the source electrode 407 and the drain electrode 409 may be composed of gold or a gold alloy.

The length 435 of the source electrode 407 and the length 441 of the drain electrode 409 are depicted as being longer than the lengths 423 and 442 of the first end section and the second end section of the central section 411, respectively. It may be desirable for the lengths 435 and 441 to be longer to ensure a solid and/or reliable electrical connection with the first end and the second end. In other embodiments, the lengths 435 and 441 may be the same as the lengths 423 and 442 for simplicity of manufacture and/or to fit more indium oxide field-effect transistors on a given substrate 401.

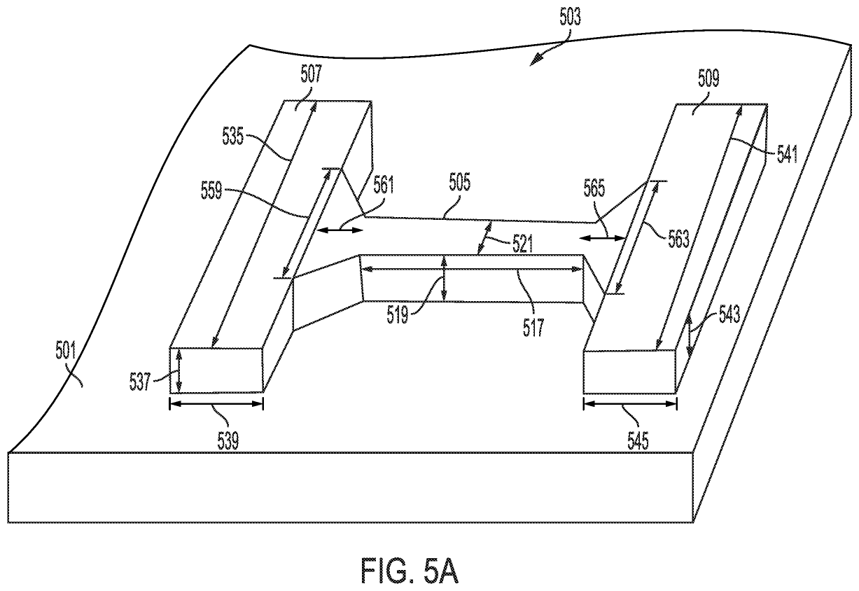

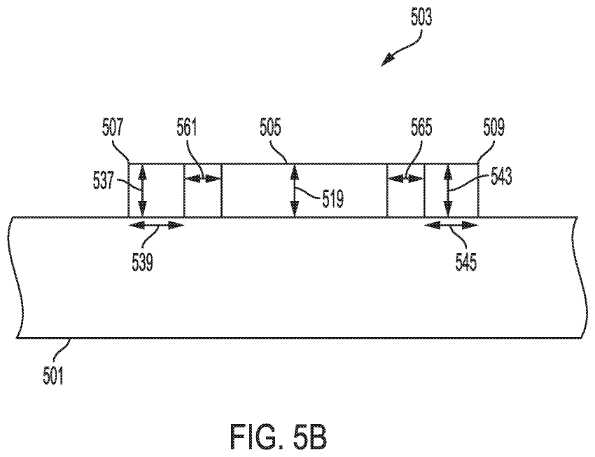

FIGS. 5A-5B show yet another alternate embodiment of an indium oxide field-effect transistor. FIG. 5A illustrates a perspective view and FIG. 5B illustrates a side view. The indium oxide field-effect transistor 503 includes a nanoribbon 505, a source electrode 507, and a drain electrode 509 located on a substrate 501.

The nanoribbon 505 includes a central section, a first end section, and a second end section. The shape of the first end section and the second end section have a transitional shape, as compared to the shape of the nanoribbon 105 of FIG. 1B, which meet the electrodes at right angles (or substantially similar to right angles). The nanoribbon 505 is composed of indium oxide. The central section has a length 517, a thickness 519, and a width 521.

The first end section contacts the source electrode 507 and has a width 559 where the nanoribbon 505 contacts the source electrode 507. The second end section contacts the drain electrode 509 and has a width 563 where the nanoribbon 505 contacts the drain electrode 509. The first end section and second end section have a thickness similar to that of the central section. The first end section may have a length 561 and the second end section may have a length 565 (as shown in FIG. 5B).

The source electrode 507 has a length 535, a thickness 537, and a width 539. The drain electrode 509 has a length 541, a thickness 543, and a width 545. The thicknesses 537, 519 and 543 may all be the same (as shown in FIG. 5B). The source electrode 507 and the drain electrode 509 are depicted as having a rectangular cross-section. However, other cross-sectional geometries may be used, for example, a truncated cylinder, a rounded square, or a rounded rectangle.

The source electrode 507 and the drain electrode 509 are composed of a conductive material. The conductive material may be any element or composition that is capable of electrical conduction. For example, the source electrode 507 and the drain electrode 509 may be composed of gold or a gold alloy.

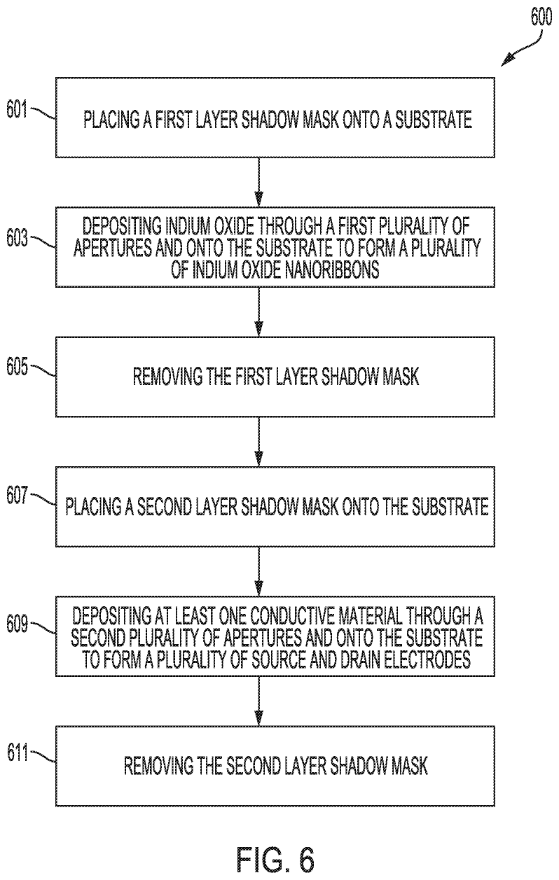

FIG. 6 illustrates a process of fabricating an array of indium oxide field-effect transistors (e.g., indium oxide field-effect transistors 203).

A first layer shadow mask (e.g., first layer shadow mask 202) is positioned over a substrate (step 601). The substrate may be composed of silicon, PET, or glass. In some embodiments, the substrate may be composed of silicon with a layer of silicon oxide on its surface.

The first layer shadow mask includes a plurality of apertures (e.g., plurality of apertures 204). The plurality of apertures are in the shape and/or form of the nanoribbons (e.g., nanoribbons 205). The first layer shadow mask may be in direct contact with the substrate when it is placed on top. In other embodiments, the first layer shadow mask may be separated from the substrate by a distance when the first layer shadow mask is placed on top of the substrate.

Indium oxide is deposited over the first layer shadow mask through the first plurality of apertures and onto the substrate to form the plurality of nanoribbons (step 603). The deposition of the indium oxide may be performed using radio frequency sputtering. However, other methods of coating may be used, for example, cathodic arc deposition, pulsed laser deposition, direct ion beam deposition, plasma-enhanced chemical vapor deposition, chemical vapor deposition, or ion beam sputtering.

After the indium oxide is deposited onto the substrate, the first layer shadow mask is removed (step 605).

A second layer shadow mask (e.g., second layer shadow mask 206) is positioned over the substrate (step 607). The second layer shadow mask includes a second plurality of apertures (e.g., second plurality of apertures 208). The plurality of apertures are in the shape and/or form of the plurality of source electrodes and drain electrodes (e.g., source electrodes 207 and drain electrodes 209). The second layer shadow mask may be in direct contact with the substrate when it is placed on top. In other embodiments, the second layer shadow mask may be separated from the substrate by a distance when the second layer shadow mask is placed on top of the substrate.

A conductive material (e.g., conductive material 210) is deposited over the second layer shadow mask through the second plurality of apertures and onto the substrate to form the plurality of source and drain electrodes (step 609). The deposition of the conductive material may be performed using electron beam evaporation. However, other methods of coating may be used, for example, cathodic arc deposition, pulsed laser deposition, direct ion beam deposition, plasma-enhanced chemical vapor deposition, chemical vapor deposition, or ion beam sputtering.

In some embodiments, an adhesion layer may be deposited onto the substrate before the conductive material is deposited in order to enhance adhesion of the conductive material to the substrate. After the conductive material is deposited onto the substrate, the second layer shadow mask is removed (step 611).

In some embodiments, the steps 601-605 are performed before steps 607-611, and in other embodiments, the steps 607-611 are performed before steps 601-605.

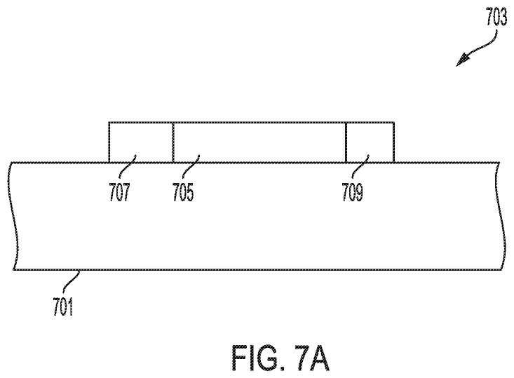

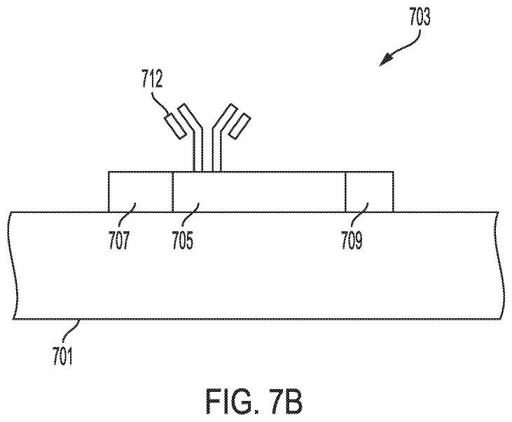

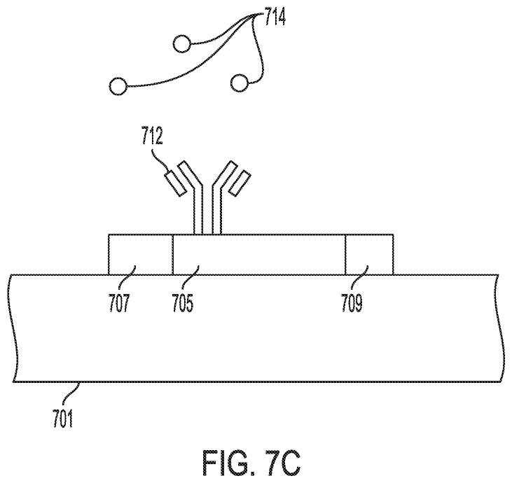

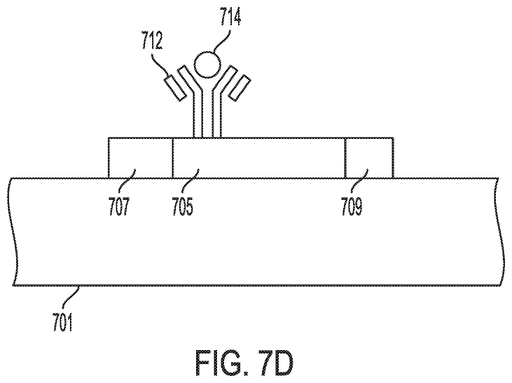

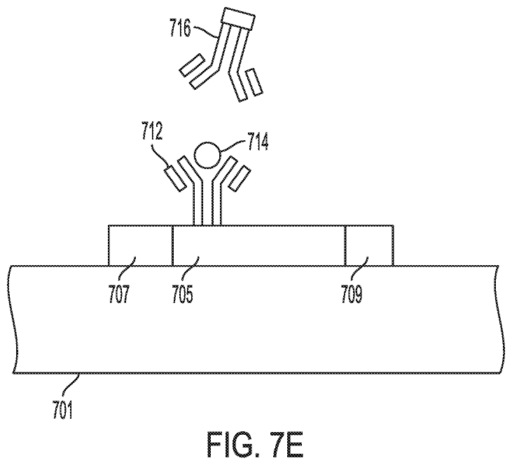

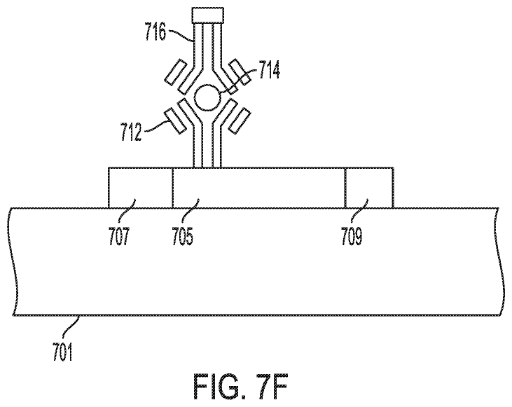

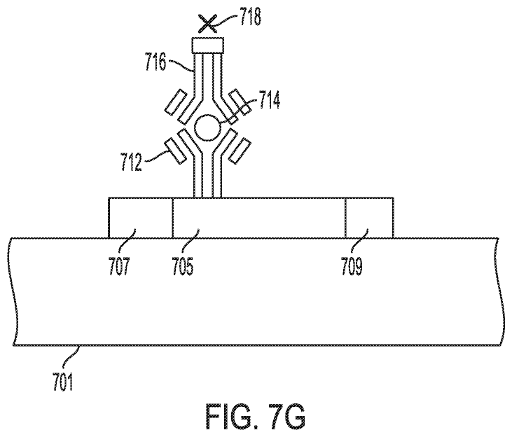

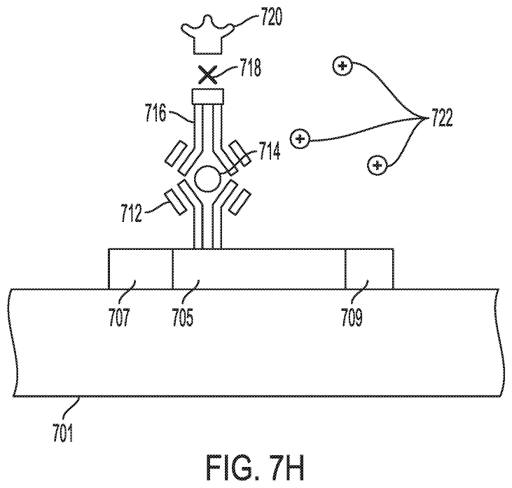

FIGS. 7A-7H illustrate a method for using an indium oxide field-effect transistor 703 as a detector. Indium oxide field-effect transistor 703 is similar to indium oxide field-effect transistor 103, and like parts are numbered similarly.

As shown in FIG. 7A, the indium oxide field-effect transistor 703 has a source electrode 707, a nanoribbon 705, and a drain electrode 709. The indium oxide field-effect transistor 703 is located on a substrate 701. Phosphonic acid may be applied to the nanoribbon 705.

As shown in FIG. 7B, a capture antibody 712 is attached to the nanoribbon 705. The capture antibody 712 corresponds to a biomarker. Preparing the nanoribbon 705 with capture antibodies may include applying a solution containing a plurality of capture antibodies to a surface of the nanoribbon 705, such that at least one capture antibody 712 attaches to the nanoribbon 705. The nanoribbon 705 may then be washed to remove unbound capture antibodies.

A fluid sample containing at least one biomarker is applied to the nanoribbon. FIG. 7C illustrates biomarkers 714 near the capture antibody 712.

FIG. 7D illustrates the capture antibody 712 connected to a biomarker 714. The nanoribbon 705 may then be washed to remove biomarkers that did not bind to the capture antibodies.

Secondary antibodies are exposed to the indium oxide field-effect transistor 703. FIG. 7E illustrates a secondary antibody 716 near the biomarker 714 and the capture antibody 712. The secondary antibody 716 corresponds to the biomarker 714.

FIG. 7F illustrates the secondary antibody 716 connected to the biomarker 714, which is connected to the capture antibody 712, which is connected to the nanoribbon 705 of the indium oxide field-effect transistor 703. The nanoribbon 705 may then be washed to remove secondary antibodies that did not bind to the biomarkers.

A protein solution 718 is applied, and bonds to the secondary antibody 716, as shown in FIG. 7G. In some embodiments, the protein solution contains streptavidin. In some embodiments, a blocking solution (e.g., bovine serum albumin) is applied to prevent nonspecific protein adsorption to the surface of the nanoribbon 705.

A reactive solution 720 is applied, and bonds to the protein solution 718, as shown in FIG. 7H. The reactive solution 720 causes an increase in the pH of the solution due to the reduction of positive hydrogen ions 722 and the surface potential. The increase in negative surface charges is responsible for the decrease in conduction of the n-type In₂O₃ nanoribbon FETs. The pH change is easily detected by the In₂O₃ nanoribbon sensors because the amount of charges released during the pH increase is very large. It is much higher than the amount of charges transferred during the direct binding between the antigens and the capture antibodies. This amplifies the detection signal and allows the sensor to detect very low concentrations of the antigen. The change in pH of the solution causes a detectible change in electrical current of the indium oxide field-effect transistor.

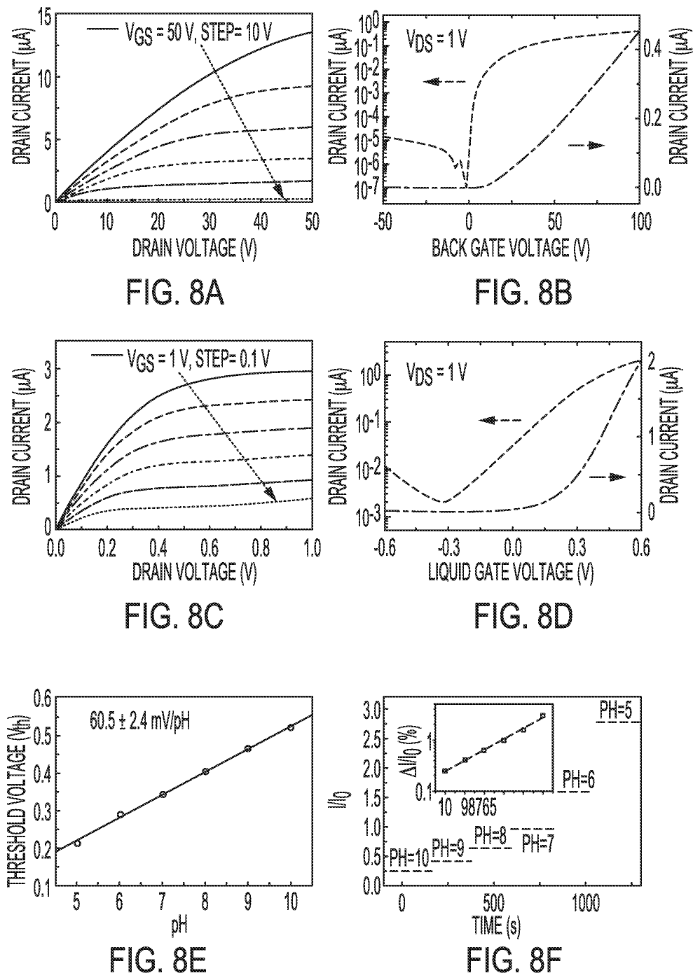

The electrical characterization of the devices are shown in FIGS. 8A-8F. The electrical characterizations were first carried out in ambient environment by measuring the output and transfer characteristics as a function of drain and back gate voltages. FIGS. 8A and 8B show family curves of drain current-drain voltage (I_{DS}−V_{DS}) and drain current-gate voltage (I_{DS}−V_{GS}) in ambient environment with drain voltage fixed at 1V, respectively. High back gate voltage may be used to turn on the device due to the presence of very thick back gate oxide. The output characteristics of the FET devices illustrate n-type transistor behavior with good saturation, and the In₂O₃ FETs show high field-effect mobilities (μₛₐₜ) of 13.09±1.39 cm²V⁻¹ S⁻¹ (averaged over 50 devices) and on/off ratios (Iₒₙ/I_{off}) above 10⁷. FIG. 8A illustrates drain current as a function of drain voltage with the back gate voltage varying from 0 to 50 V in steps of 10 V. FIG. 8B illustrates drain current versus back gate voltage with drain voltage fixed at 1 V. Current is plotted in logarithmic scale in the left axis and in linear scale in right axis.

For biosensing applications, these devices may be operated in a wet environment. Hence, the devices were measured with the active channel materials immersed in a micro well filled with electrolyte solution (0.01×Phosphate Buffered Saline (PBS)). An Ag/AgCl reference electrode is used to apply bias to the electrolyte, which is referred to as a liquid gate to stably operate the biosensor. The performance of liquid-gated In₂O₃ FETs is shown in FIG. 8C (I_{DS}−V_{DS}) and FIG. 8D (I_{DS}−V_{GS}). It illustrates that the biosensor device is efficiently controlled in the wet environment, and the In₂O₃ FETs have good FET behavior with saturation and low driving voltage. FIG. 8C illustrates family curves of I_{DS}− V_{DS} measured in 0.01×PBS with liquid gate varying from 1 V to 0.5 V in steps of 0.1 V. FIG. 8D illustrates drain current versus liquid gate voltage with drain voltage fixed at 1 V, also plotted in linear and logarithmic scale. FIG. 8E illustrates a change in threshold voltage with pH range from 5 to 10, and an obtained pH sensitivity of approximately 60.5 mV/pH.

The results of experiments using the systems and methods described herein are disclosed below. To determine the pH sensitivity of the In₂O₃ FETs, six devices were randomly selected from the wafer, and their response to pH solutions were recorded. The pH sensing is based on the protonation/deprotonation of the OH⁻ groups on the surface due to the pH value of the electrolyte, and thereby changes in local FET electric fields, which cause changes in the conductance and current. The shift in threshold voltage, which has been calculated using the extrapolation in the saturation region was found to be 60.5±2.44 mV/pH at room temperature, close to the ideal result of 59.1 mV/pH at 25° C. FIG. 8F shows the real-time sensing response of an unfunctionalized In₂O₃ FET to standard pH calibration solutions. The initial current Iₒ was obtained by using PBS to stabilize the device, and then the PBS buffer was sequentially changed to commercial pH buffer solutions ranging from pH 10 to pH 5. The drain current responded quickly and log-linearly to each pH buffer.

Direct electrical detection of biomolecules in their physiological environment is often impeded by the Debye screening from the high salt concentration in the sample solutions. Sandwich enzyme-linked immunosorbent assay (ELISA), on the other hand, detects signals associated with the reactions between a test solution and the conjugated enzymes on secondary antibodies instead of the biomarker. The sandwiched structure not only overcomes the Debye screening from salts in the fluid, but also incorporates an amplification scheme to improve the signal-to-noise ratio (SNR), which can be much higher than direct analyte detection without amplification, especially when the amount of analytes is small.

In the following In₂O₃ nanoribbon sensing experiments, an electronic ELISA technique was applied that uses pH change due to urease enzyme activities as the amplification signal. The schematic diagram depicting the electronic ELISA process are similar to the process described in FIGS. 7A-7F.

Prior to using In₂O₃ FET biosensors for biomarker detection, the surfaces were treated with phosphonic acid to confer phosphonic linker molecules to the indium oxide surface. Subsequently, the devices were functionalized with N-(3-Dimethylaminopropyl)-N′-ethylcarbodiimide hydrochloride/N-Hydroxysuccinimide (EDC/NHS) chemistry to immobilize the capture antibodies on the surface of In₂O₃ FETs, as described herein. This was followed by a washing step that removed unbound capture antibodies (all binding steps described below were followed by three times washes). A bovine serum albumin (BSA) solution was used to prevent nonspecific protein adsorption to the chip and reservoir sidewalls, which is a typical blocking step used in conventional colorimetric ELISA protocols to minimize nonspecific binding. This was followed by introducing known concentrations of the antigen-containing samples to the sensor for antigen-antibody binding. The biomarkers were contained either within the physiological fluid sample of the patient or in a solution of buffer for experimental purposes. The biomarkers were subsequently captured by the antibodies, and any unbound ones were washed off. Next, a solution of biotinylated secondary antibodies which is also specific to the cardiac biomarker was introduced to the sensors using incubation, and the secondary antibodies bound themselves to the biomarkers. After rinsing out unbound biotinylated antibody, streptavidin solution in PBS was introduced. The biotin end of the secondary antibody group was used to bind to a streptavidin, which in turn was bound to a biotinylated urease, the last solution to incubate the sensor.

When a solution of urea is introduced to the nanoribbon sensor surface with this sandwich structure, the urea causes an increase in the pH of the solution due to consumption of hydrogen ions according to the following reaction.

$$Urea + 2 {H}_{2} O + {H}^{+} {\to }^{ Urease } 2 {NH}_{4}^{+} + {HCO}_{3}^{-}$$

The urease deprotonates free hydroxyl groups on the surface of In₂O₃ nanoribbon, and the pH increases due to the reduction of positive hydrogen ions and the surface potential. The increase in negative surface charges is responsible for the decrease in conduction of the n-type In₂O₃ nanoribbon FETs. The pH change is easily detected by the In₂O₃ nanoribbon sensors because the amount of charges released during the pH increase is very large. It is much higher than the amount of charges transferred during the direct binding between the antigens and the capture antibodies. This amplifies the detection signal and allows the sensor to detect very low concentrations of the antigen. Furthermore, the solution for the pH detection step is independent of the fluid containing the biomarker, since the solutions are rinsed out after each step. This allows cardiac biomarkers to be collected in physiological samples such as whole blood without the limitation of the Debye screening effect.

Troponin, a Food and Drug Administrative (FDA) approved biomarker for AMI, is the biomarker of choice for evaluating chest pain patients for possible heart attack. Troponin I and T are released to blood streams due to the death of cardiac muscle cells; therefore, troponin I and T are not present in the blood of healthy people. Elevated blood troponin levels have a positive correlation to the risk of death in the heart disease patients, and the biomarker is a good guide for identifying patients for certain types of treatment. The 99th percentile of a reference decision limit (medical decision cutoff) for cardiac troponin (cTn) assays is over 40 pg/ml. In the first biomarker detection experiment, troponin I was used as the model cardiac biomarker to demonstrate that the In₂O₃ nanoribbon biosensors can be used to optimize the electronic ELISA assay turnaround time by shortening the incubation of the cardiac biomarkers to 30 minutes in total. The incubation time of target analytes, biotinylated secondary antibodies, streptavidin and biotinylated urease enzymes were 10, 10, 5, and 5 minutes, respectively.

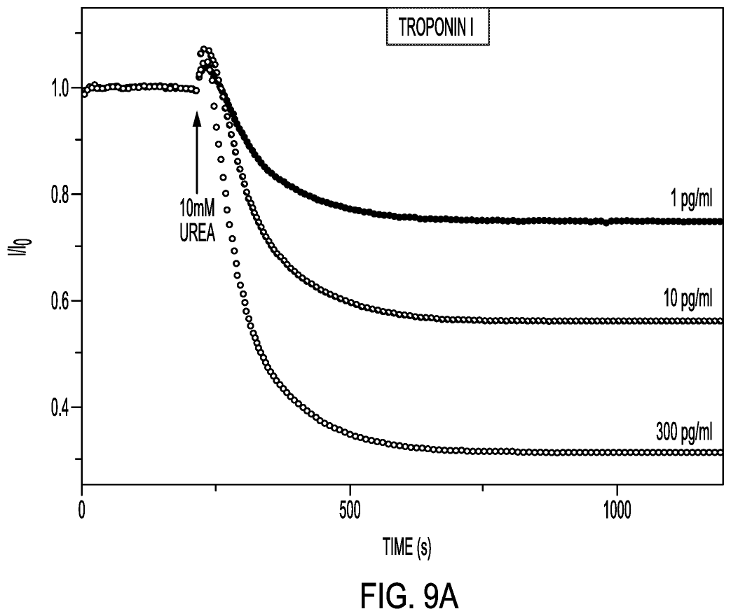

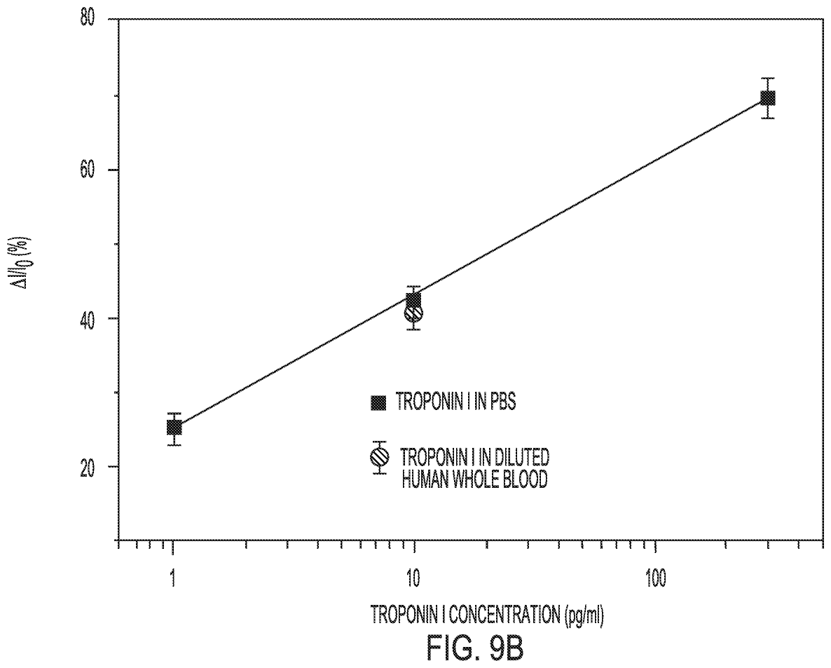

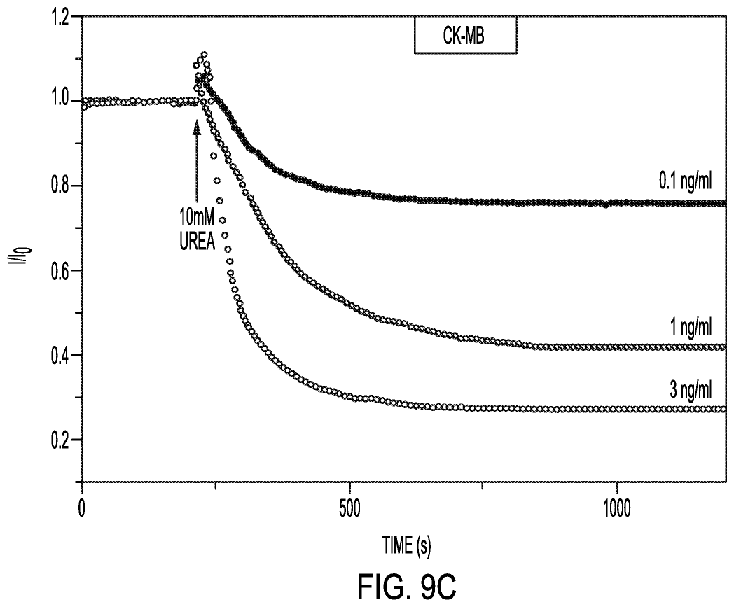

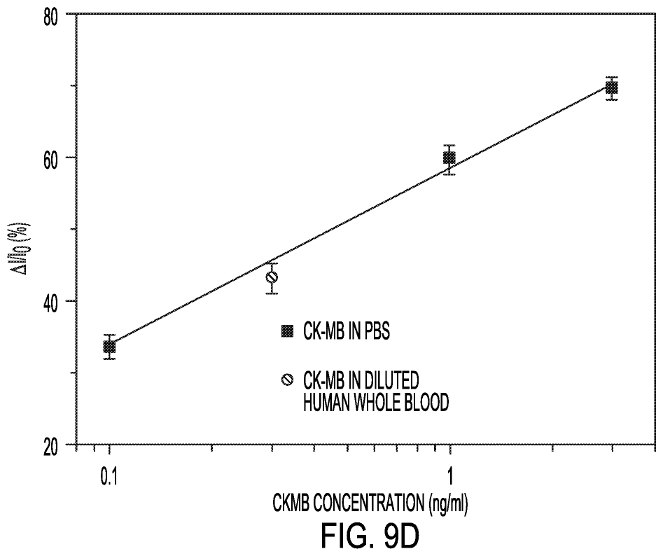

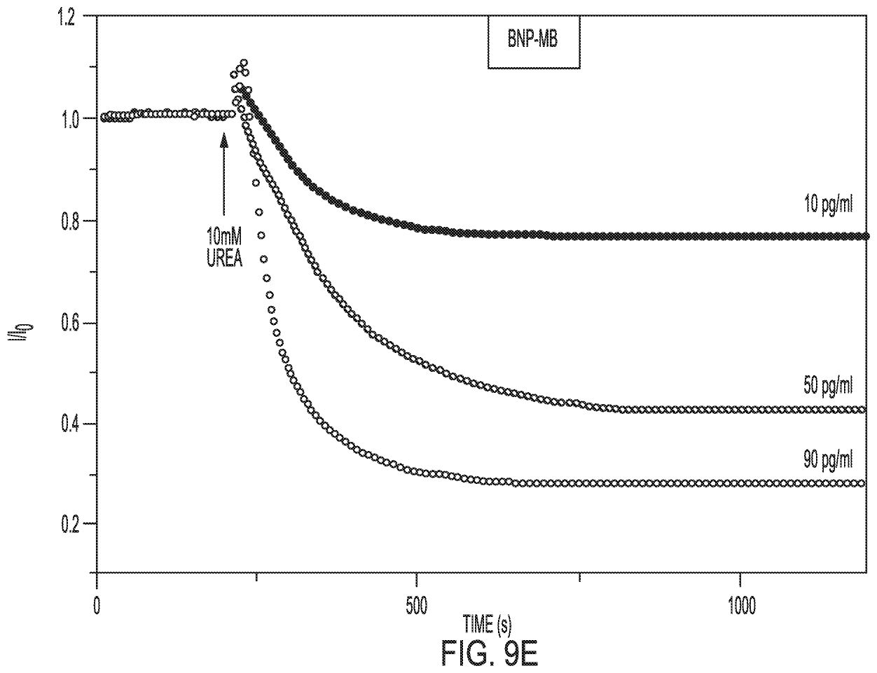

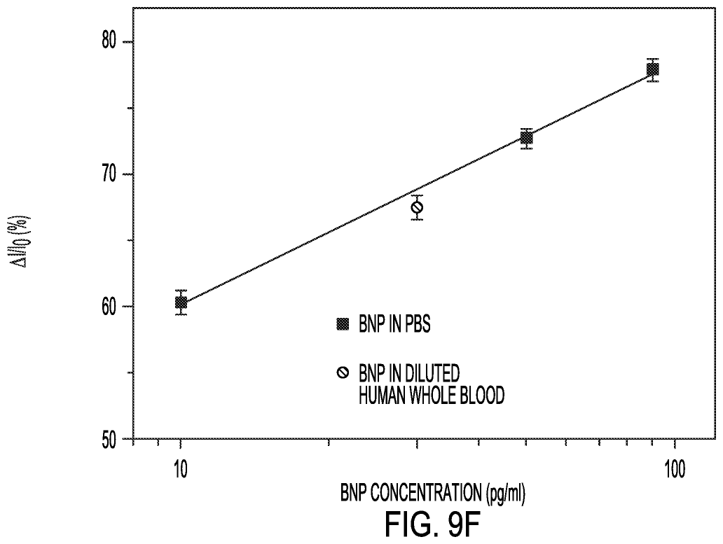

Experiments were performed with known concentration of cardiac Troponin I (cTnI) in 1×PBS, namely 1, 10 and 300 pg/ml, to build a standard curve covering the beginning of the second quartile for non-AMI patients to the median of AMI patients. FIG. 9A illustrates real-time sensing results of 1 pg/ml, 10 pg/ml and 300 pg/ml of cTnI antigens in 1×PBS buffer. At time t=0 in FIG. 9A, the devices were rinsed with and submerged in 0.01×PBS buffer when the baseline current was taken. The buffer was then replaced with 10 mM urea in 0.01×PBS around 200 s as indicated by the arrow. It shows the real-time responses from 3 sensors when the urea solution was introduced into the sensing chamber that was previously incubated in 1 pg/ml, 10 pg/ml and 300 pg/ml of cardiac troponin I (cTnI) in 100 μl of 1×PBS buffer. The urease-urea interaction drastically reduces the device conductance by 25.3%, 42.5% and 69.5% of the baseline signal, respectively.

FIG. 9B illustrates average sensing results of 3 devices from 3 concentrations of cTnI proteins in 1×PBS buffer marked as black square and 1 concentration of troponin I in diluted human whole blood marked as a dot. Error bars represent standard deviations of 3 devices. Each data point was calculated from three sensors monitored simultaneously during the experiment. The sensing response decreases exponentially upon the decrease in the concentration of cTnI target molecules. The current of the In₂O₃ nanoribbon sensor drops to about 42% of the baseline at a troponin concentration of 10 pg/ml and 25% at a concentration of 1 pg/ml. The sensitivity corresponds to about 17% conduction change per decade of biomarker concentration change. This is beneficial for covering a large range of concentrations for biomarkers like cTnI whose elevation in AMI patients is high. The pH changes between the buffer solutions used for the baseline and the final solutions in the sensing chamber was measured to be 0.17, 0.87 and 2.17, respectively, by a commercial Mettler Toledo pH meter. These increases in pH are consistent with the decreases in conduction of the In₂O₃ nanoribbon devices. Moreover, the total sample-collection-to-result time is around 45 mins, which meets the expectation of 1 hour for practical use in diagnosis of myocardial infarction.

In addition to cTnI, the blood biomarker Creatine kinase-MB (CK-MB) has long been used for AMI detection. Including the detection of CK-MB can improve early diagnosis of AMI, since the level of CK-MB increases within 2 to 4 hours after cardiac muscle injury. The CK-MB level in the blood is relatively high compared to other biomarkers, with an interquartile range level of non-AMI patients at 0.6 ng/ml to 1.7 ng/ml, and that of AMI patients from 1.5 ng/ml to 10.5 ng/ml. Thus, the detection of CK-MB must be able to distinguish the concentration change less than one order of magnitude for effective diagnosis. The sensing was repeated for 0.1, 1 and 3 ng/ml.

FIG. 9C illustrates real-time sensing results of 0.1 ng/ml, 1 ng/ml and 3 ng/ml of CK-MB proteins in 1×PBS buffer. Heart failure is strongly indicated when the blood sample has 30 ng/ml CK-MB before dilution or 3 ng/ml with 10×dilution. The average of data from 3 sensors for each concentration is plotted in FIG. 9D with standard deviations plotted as the error bars, and the pHs change between At 0.1 ng/ml, the current is approximately 33% of the baseline, and at 1 ng/ml, the current is approximately 60% of the baseline, yielding a difference equivalent to 27% of the baseline for a concentration difference of a decade. This large sensing response enable detection of minute changes in concentration, such as from 250 pg/ml to 300 pg/ml or 2.5 ng/ml to 3 ng/ml before 10×dilution. In addition, the small device-to-device signal standard deviation makes readout at this precision possible using the In₂O₃ nanoribbon sensor platform.

Besides cTnI and CK-MB, B-type natriuretic peptide (BNP) is also associated with heart failure and has been shown to substantially improve AMI diagnosis when included in a multiple cardiac biomarker panel. More importantly, for blood samples taken when chest pain patients first arrive at the emergency department, BNP is shown to have quicker response for AMI diagnosis than other cardiac biomarkers such as CK-MB and troponin, which do not elevate until at least 2 hours after the onset of AMI symptoms. In fact, even when a patient's troponin level is normal, a BNP concentration greater than 100 pg/ml is a good indicator of AMI. BNP higher than 900 pg/ml is considered severe heart failure.

To simulate quantitative detection of BNP in patients' blood, a standard calibration curve with known concentrations of BNP in buffer was first obtained. As shown in FIG. 9E, BNP concentrations of 10 pg/ml, 50 pg/ml, and 90 pg/ml were targeted. For each of the 3 concentrations, 3 In₂O₃ nanoribbon sensors were used in the electronic ELISA assay as described herein. For the smallest concentration of 10 pg/ml, the current drops to approximately 60% of the baseline after the introduction of 10 mM Urea solution. For the 50 pg/ml and the 90 pg/ml detection, the current drops to 72% and 77% of the baseline, respectively. The average and the standard deviation for each of the three concentrations are plotted in FIG. 9F. In logarithmic scale, the linear fitting has an R-squared value of 0.9978, suggesting a good fit for the BNP concentration calibration curve.

Detection of cardiac biomarkers in whole blood is essential to POC sensor platforms used for situations where complicated patient blood processing is not possible and defeats the purpose of fast, cheap, and convenient disease testing. The main problems for FET sensor detection caused by whole blood are the nonspecific binding of non-target proteins and the Debye length screening from salts. Recent efforts to process whole blood for FET sensors have been demonstrated using a microfluidic chip, desalting columns, and filtration. The systems and methods described herein have made improvements and demonstrated that by applying electronic ELISA assay on In₂O₃ nanoribbon sensors, cardiac biomarkers such as cTnI, CK-MB, BNP in whole blood can be detected without any sample processing at all, as described below.

Cardiac biomarkers, cTnI, CK-MB and BNP, were spiked with healthy human whole blood (purchased from Innovative Research) to simulate an AMI patient sample with a cTnI concentration 100 pg/ml, a CK-MB concentration 3 ng/ml and a BNP concentration of 300 pg/ml, all indicating mild heart failure. Because human whole blood is very viscous, which may affect the sensing results, a real patient sample would be first diluted 10 times to be detected by the nanoribbon sensor. This sample dilution is not due to the difficulties in ionic screening and does not filter out any non-specific proteins or blood cells. To simulate this sample preparation, 100 μl of healthy whole blood was first diluted with 1×PBS to 930 μl. Then 10 μl of 1 ng/ml cTnI, 50 μl of 6 ng/ml CK-MB and 10 μl of 3 ng/ml BNP in 1×PBS was added to the diluted whole blood to simulate 10 pg/ml cTnI, 0.3 ng/ml CK-MB, 30 pg/ml BNP in 10×diluted whole blood. This sample was used to incubate the nanoribbon sensors prepared with capture antibodies. The remaining steps of the electronic ELISA assay followed those described previously herein.

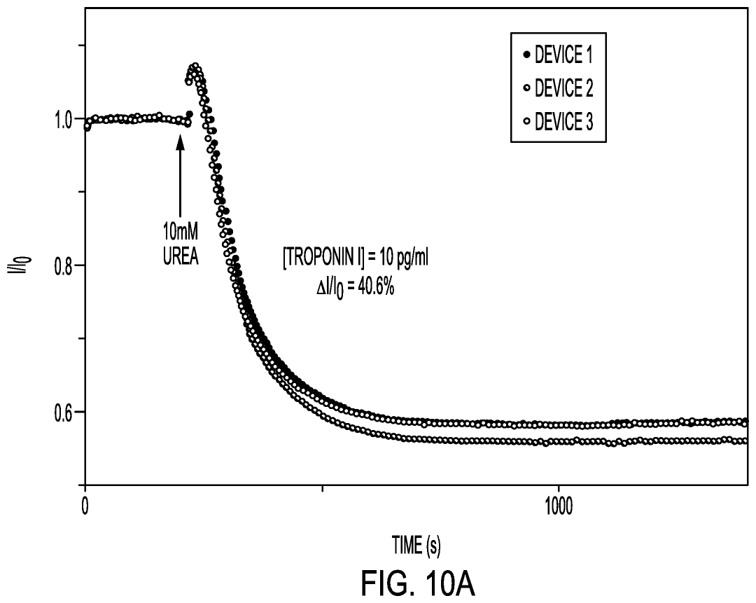

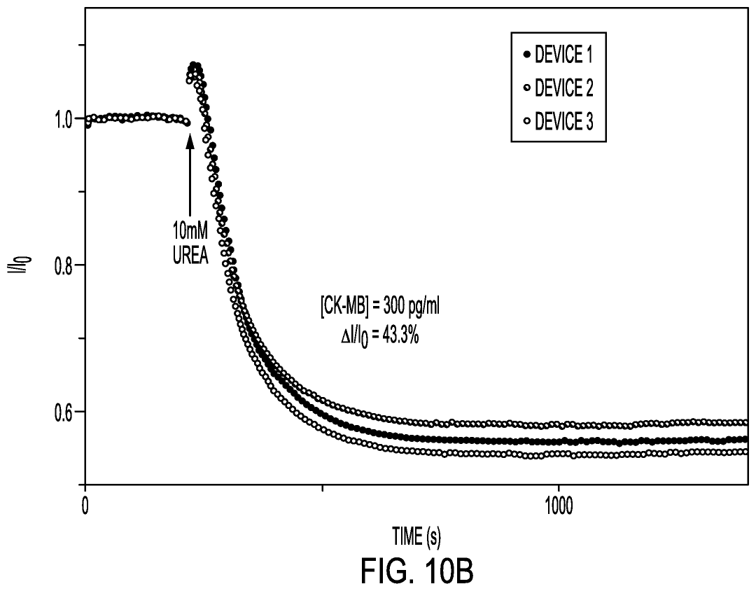

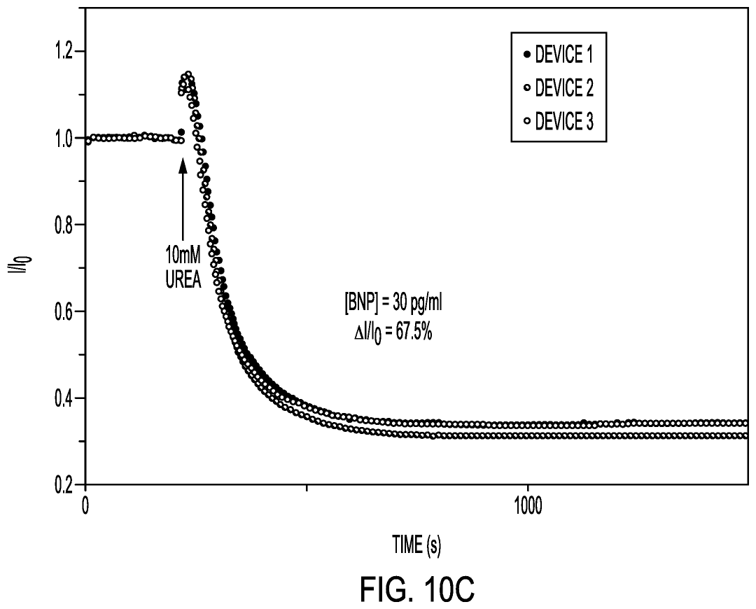

FIGS. 10A-10C show the real-time signal when 10 mM of urea in 0.01×PBS is introduced to each sensor. The current drop is 40.6% for 10 pg/ml cTnI, 43.3% for 0.3 ng/ml CK-MB and 67.5% for 30 pg/ml BNP. FIG. 10A illustrates real-time sensing results of 10 pg/ml of cTnI in 10×diluted human whole blood, and the averaged results were plotted as a dot in FIG. 9B. FIG. 10B illustrates real-time sensing results of 300 pg/ml of CK-MB in 10×diluted human whole blood, and the results were plotted as a dot in FIG. 9D. FIG. 10C illustrates real-time sensing results of 30 pg/ml of BNP in 10×diluted human whole blood, and the results were plotted as red dot in FIG. 9F. In FIGS. 9B, 9D, and 9F, the average responses of the 3 In₂O₃ nanoribbon sensors for this detection are placed on the calibration curve as dots. The graph shows that the deviation of the detection signal from the calibration curve are all below 5%. This falls within the device-to-device variation and is expected for the experiment.

Since the cardiac biomarkers concentration elevate in AMI patients, it is important for diagnosis and treatment to obtain the trend in the cardiac biomarker concentration in the hours after patient's arrival. The reusability of the In₂O₃ nano-biosensor can give results every hour when the sensing is repeated. In addition, the reusability of the biosensors is also cost-effective. Following the regeneration process of antibodies and antigens, 50 mM NaOH was applied as regeneration buffer (from GE healthcare) to a sensor that had already been used for cTnI biomarker sensing. When rinsing the sensors with washing buffer, the proteins will anti-bind the capture antibody. After rinsing with 1×PBS buffer, the proteins will all be washed away, leaving the antibody still active and bond to the surface of the sensor. To demonstrate the sensors can still work well after washing, incubation was started with samples containing cTnI biomarkers and the electronic ELISA process described herein was performed.

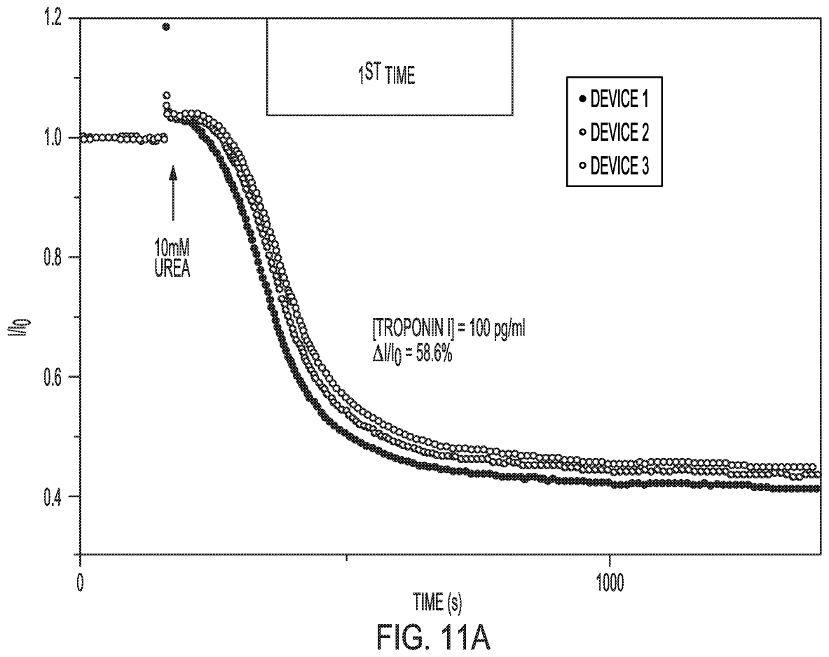

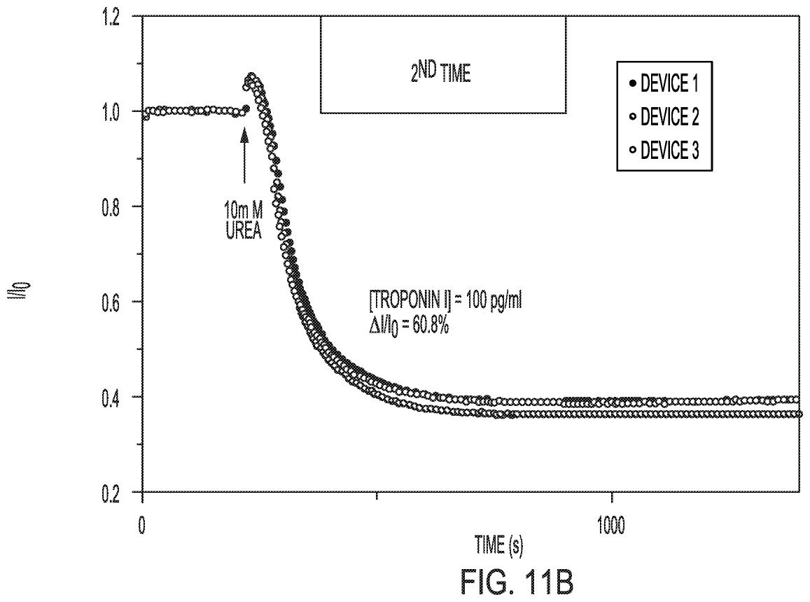

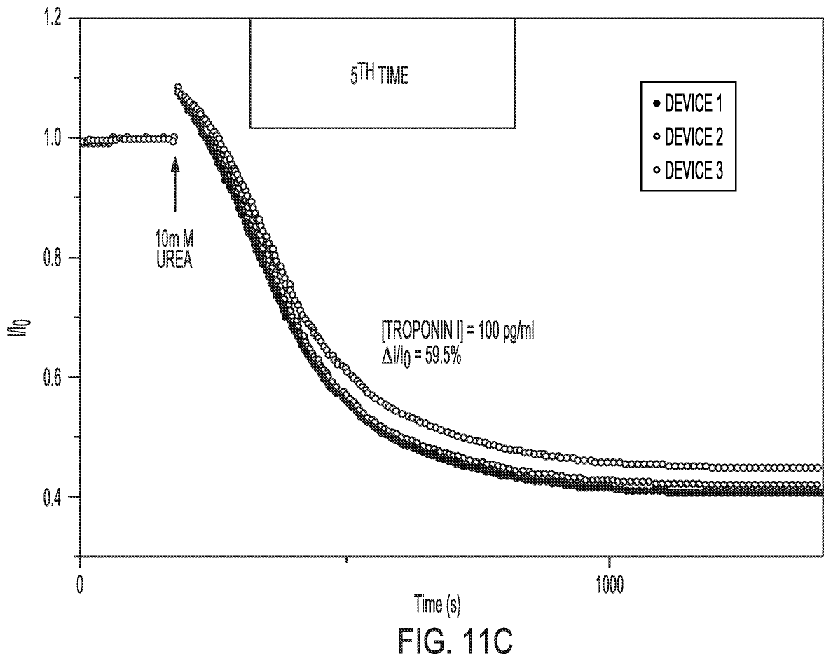

FIGS. 11A-11C show the sensing results for the first time, the second time and the fifth time, respectively. They all fall around 60%, which indicates the regeneration process can efficiently wash proteins away and leaves sufficient capture antibodies for reusability. FIG. 11A illustrates real-time responses of 100 pg/ml cTnI proteins in 1×PBS buffer. FIG. 11B illustrates real-time sensing responses from the same concentration of cTnI and the same devices after regeneration. FIG. 11C illustrates real-time response of the same sensors after 3 more cycles of regeneration and sensing process.

In a production setting, further improvements can be made for even better uniformity by monitoring the nanoribbon film thickness after sputtering and chemical modification to reduce the device-to-device variation down a fraction of a percentage. Such highly uniform batches of sensors can give good statistical confidence for their reported biomarker concentrations. This confidence level combined with a turnaround time of 45 minutes is a good basis for improving current POC devices for cardiac marker detection in an emergency situation. Moreover, the platform can be integrated with other electronic components for better data analysis.

The details of the experimental methods are described below.

Materials: 3 inch 500 nm SiO2 on Si wafers was purchased from SQI. Au and Ti for metal sources of electron beam evaporation and an indium oxide (In₂O₃) sputtering target with purity of 99.99% were obtained from Plasmaterials. 3-Phosphonopropioninc acid with purity of 94%, N-(3-Dimethylaminopropyl)-N′-ethylcarbodiimide hydrochloride (EDC) with purity of 98%, and N-Hydroxysuccinimide (NHS) with purity of 98% were purchased from Sigma Aldrich. Shadow mask for patterning were purchased from Photo Science. Troponin I monoclonal antibodies, Troponin I proteins, biotinylated Troponin I monoclonal antibodies, CK-MB antibodies, CK-MB proteins, biotinylated CK-MB antibodies, BNP antibodies and BNP proteins were purchased from Fitzgerald Industries. Biotinylated BNP polyclonal antibodies were purchased from Abcam.

Shadow Mask Fabrication method: SiO₂/Si wafer was rinsed with acetone and isopropyl alcohol before dried in nitrogen stream before the fabrication process. After solvent cleaning, the SiO₂/Si substrate was placed on a hot plate at 120° C. for 5 minutes to repel all solvent residual and cool down in room temperature. After cleaning process the first shadow mask was attached to the SiO₂/Si wafer to pattern the channel area. Then the In₂O₃ ribbons were deposited by RF sputtering (by Denton Discovery 550 sputtering system in NRF). By simply remove the shadow mask, we got well defined nanoribbons. The source and drain electrodes were defined by the second shadow mask. After using aligner to pattern the source and drain area, we attached the shadow mask and the substrate. Then followed with deposition of 1 nm Ti and 50 nm Au by employ electron beam evaporation. After deposition, the shadow mask was removed and yield pristine surface.

Characterization: Optical microscopy images were taken with Olympus microscope. Atomic force microscopy imaging was performed on DI 3100 Digital Instruments under tapping mode. The SEM images were taken with a Hitachi S-4800 field emission scanning electron microscope. Electrical characteristics in ambient environment of the In₂O₃ TFTs were measured with an Agilent 4156B Precision semiconductor parameter analyzer in ambient environment. Electrical characteristics in wet environment and sensing results were measured with an Agilent 1500B semiconductor analyzer.

In conclusion, the fabrication of highly uniform and scalable In₂O₃ nanoribbon biosensor chips using two simple shadow masks to define the position and dimension of metal electrodes and nanoribbons was demonstrated, and the devices showed outstanding performance. Furthermore, In₂O₃ nanoribbon devices show good electrical performance in the aqueous condition when gate voltage is applied through the liquid gate electrode. In addition, the In₂O₃ nanoribbon devices show good performance in pH sensing experiment with change in conduction by a factor of 12 when pH is reduced from 10 to 5. Through all the sensing experiments, it has been demonstrated that In₂O₃ nanoribbon biosensors fabricated by shadow masks can be used to quantitatively detect 3 cardiac biomarkers within the concentrations relevant to clinical diagnosis with the turnaround time of approximately 45 minutes. Tests were further demonstrated using spiked cardiac biomarkers in diluted human whole blood, with results consistent with the calibration curve established using PBS buffer. Lastly, by applying a regeneration buffer to the used sensor surfaces to anti-bond the antigen-antibody conjugation and repeating the sensing experiments, the reusability of the In₂O₃ nanoribbon biosensors with very small variation of each sensing results was demonstrated.

The foregoing description of the disclosed example embodiments is provided to enable any person of ordinary skill in the art to make or use the present invention. Various modifications to these examples will be readily apparent to those of ordinary skill in the art, and the principles disclosed herein may be applied to other examples without departing from the spirit or scope of the present invention. The described embodiments are to be considered in all respects only as illustrative and not restrictive and the scope of the invention is, therefore, indicated by the following claims rather than by the foregoing description. All changes which come within the meaning and range of equivalency of the claims are to be embraced within their scope.

What is claimed is:

1. A method for using an indium oxide field-effect transistor, the method comprising:
applying phosphonic acid to a nanoribbon of the indium oxide field-effect transistor;
preparing the nanoribbon with capture antibodies corresponding to a biomarker;
applying a fluid sample containing at least one biomarker to the nanoribbon;
preparing the nanoribbon with secondary antibodies corresponding to the biomarker;
applying a protein solution to the nanoribbon; and
detecting the presence of the at least one biomarker when a reactive solution is applied to the nanoribbon.

2. The method of claim 1, wherein preparing the nanoribbon with capture antibodies comprises applying a solution containing a plurality of capture antibodies to a surface of the nanoribbon, at least one capture antibody within the plurality of capture antibodies binding to the surface of the nanoribbon.

3. The method of claim 2, further comprising washing the nanoribbon to remove unbound capture antibodies.

4. The method of claim 3, further comprising applying a blocking solution configured to prevent nonspecific protein adsorption to the surface of the nanoribbon.

5. The method of claim 4, further comprising washing the nanoribbon to remove biomarkers that did not bind to the capture antibodies.

6. The method of claim 5, further comprising washing the nanoribbon to remove secondary antibodies that did not bind to the biomarkers.

7. The method of claim 6, wherein the reactive solution has a pH and the application of the reactive solution to the surface of the nanoribbon causes the pH of the solution to change.

8. The method of claim 7, wherein the change in pH of the solution causes a detectable change in electrical current of the indium oxide field-effect transistor.

9. The method of claim 1, wherein the protein solution contains streptavidin.

10. The method of claim 1, wherein the secondary antibodies are biotinylated.

11. The method of claim 1, further comprising immersing the indium oxide field-effect transistor in an electrolyte solution.

12. The method of claim 1, further comprising detecting signal from the indium oxide field-effect transistor using sandwiched enzyme-linked immunosorbent assay (ELISA).

13. The method of claim 12, wherein the sandwiched ELISA overcomes Debye screening from salts in the fluid, and incorporates an amplification scheme to improve the signal-to-noise ratio (SNR) compared to direct analyte detection without amplification.

14. The method of claim 1, wherein the biomarker is troponin.

15. The method of claim 1, wherein the biomarker is Creatine kinase-MB.

16. The method of claim 1, wherein the biomarker is B-type natriuretic peptide.

17. The method of claim 1, wherein the presence of the biomarker is detected from whole blood.

18. The method of claim 17, further comprising diluting the whole blood.

19. The method of claim 1, wherein preparing the nanoribbon with capture antibodies comprises applying N-(3-Dimethylaminopropyl)-N′-ethylcarbodiimide hydrochloride/N-Hydroxysuccinimide (EDC/NHS) to a surface of the nanoribbon.

20. The method of claim 1, further comprising applying a regeneration buffer to the nanoribbon to reuse the nanoribbon.
